(19) United States
(12) Patent Application Publication
(10) Pub. No.: US 2003/0022249 A1
(43) Pub. Date: Jan. 30, 2003

(54) Antigen-binding fragments that recognize a subset of dendritic cells and methods of use thereof

(76) Inventors: Juergen Schmitz, Bergheim (DE); Andrzej Dzionek, Koeln (DE); David William Buck, Sussex (GB)

Correspondence Address: Randolph Ted Apple, Morrison & Foerster LLP, 755 Page Mill Road, Palo Alto, CA, 94304-1018, US

(21) Appl. No.: 10/150,440

(22) Filed: May 17, 2002

Related U.S. Application Data
(60) Provisional application No. 60/291,561, filed on May 17, 2001.

(51) Int. Cl. G01N 33/567; C12N 5/08

(52) U.S. Cl. 435/7.21; 435/372

(57) ABSTRACT

The invention provides methods of separation, enrichment and analysis of hematopoietic cell populations containing dendritic cells and subsets thereof. Compositions containing dendritic cells and methods of use of such compositions are provided.

### CROSS-REFERENCE TO RELATED APPLICATIONS

This application claims benefit of U.S. provisional application 60/291,561, filed May 17, 2001, the entire contents of which is incorporated by reference herein.

### TECHNICAL FIELD

The present invention relates to methods of using antibodies and derivatives thereof that recognize a subpopulation of dendritic cells (DCs) termed BDCA-3. Methods of use of the isolated dendritic cells include DC-based immunotherapy, characterization of various diseases and in vivo numeric DC expansion for instance with flt3-Ligand.

### BACKGROUND OF THE INVENTION

The hematopoietic development of DCs, potent antigen presenting cells (APCs), is distinct and may follow several precursor pathways some closely linked to monocytes. DCs may be derived from a lymphoid precursor. Thomas et al. (1993) J. Immunol. 150:821-834. Like in blood, there may be three distinct subsets of DCs present in the thymus: 1) plasmacytoid CD4⁺CD11c- DCs; 2) CD4⁺CD11c⁺ dendritic cells; and 3) interdigitating DCs. It has been proposed that thymic DCs and T cells arise from a common stem cell. Thomas et al. (1996) Stem Cells 14:196-206.

Generation of large numbers of DCs for potential clinical use has recently been accomplished through the in vitro culturing of progenitors with cytokines. Various strategies have been adopted to introduce antigens into dendritic cells so that they may be more effectively presented to T cells in the context of costimulation. It has also been shown that dendritic cells can influence the T cell response to antigen to follow either a humoral or systemic pathway.

T cells are unable to respond to unprocessed proteins, rather, they require accessory cells to present antigen as peptide epitopes displayed on the cell surface in conjunction with MHC molecules. Antigens generated endogenously in the cell cytoplasm are typically presented in the Class I pathway and stimulate cytotoxic T lymphocyte (CTL) reactions while exogenous protein is process in MHC Class II compartments and induce helper (CD4) T cell responses. The stimulation of naive T cells requires the presence of costimulatory molecules that act as secondary signals in the activation of primary immunity. APCs such as B cells and macrophages are typically incapable of inducing primary responses. In contrast, dendritic cells drive their potency from the constitutive unregulated expression of costimulatory, adhesion and MHC Class I and II molecules essential for the initiation of effective cellular immunity. For review see, Avigan (1999) Blood Rev. 13:51-64.

DCs are APC that are essential for initiation of primary immune responses and the development of tolerance. DCs express MHC, necessary for stimulation of naive T cell populations. The hematopoietic development of DCs is distinct and may follow several precursor pathways, some of which are closely linked to monocytes. See, for review, Avigan (1999). Different DC subsets have distinct developmental pathways. The emerging concept is that one DC subset has regulatory functions that may contribute to the induction of tolerance to self-antigens. Austyn (1998) Curr. Opin. Hematol. 5:3-15. Conversely, DCs, or a subset thereof, may also be involved in the induction of immune responses to self-proteins. It is thought that certain autoimmune responses may be due to microenvironmental tissue injury followed by local DC activation and subsequent interaction with T cells to initiate an immune response. Ibrahim et al. (1995) Immunol. Today 16:181-186.

The ability of DCs to initiate T cell responses is being used in DC cancer vaccines. Hart et al. (1999) Sem. Hematol. 36:21-25. For instance, DCs are generated in vitro from CD34⁺ cells or monocytes, pulsed with tumor-derived peptides or proteins and returned to the patient to act as APCs in cancer-specific T cell induction. Brugger et al. (1999) Ann. N.Y. Acad. Sci. 872:363-371. Animal models have demonstrated that DC tumor vaccines reverse T cell anergy and result in subsequent tumor rejection. Avigan (1999); see also, Tarte et al. (1999) Leukemia 13:653-663; Colaco (1999) Molec. Med. Today 5:14-17; Timmerman et al. (1999) Ann. Rev. Med. 50:507-529; Hart et al. (1999); Thurnher et al. (1998) Urol. Int. 61:67-71; and Hermans et al. (1998) N.Z. Med. J. 111:111-113. One approach has been to increase DCs in vivo by administration of fit-Ligand. This has the effect of compensating for VEGF-induced DC suppression. Ohm et al. (1999) J. Immunol. 163:3260-3268. DCs have been proposed for use as adjuvants in vaccination and in recombinant vaccines. Fernandez et al. (1998) Cyto. Cell. Mol. Ther. 4:53-65; and Gilboa et al. (1998) Cancer Immunol. Immunother. 46:82-87. DCs have also been proposed for use in enhancing immunity after stem cell transplantation. Brugger et al. (1999). DCs play a number of potential roles in immunology. For instance, DCs are involved in human immunodeficiency virus (HIV) infection. Zoeteweij et al. (1998) J. Biomed. Sci. 5:253-259. DCs have also been proposed as suitable for use in HIV therapy. Weissman et al. (1997) Clin. Microbiol. Rev. 10:358-367.

Studies on DCs in blood have been hampered by scarcity of the cells and the relative lack of DC-specific cell surface markers. Methods for DC isolation are based on either maturational change after a short culture period, like the acquisition of low buoyant density or the expression of DC activation/maturation antigens (CD83, CMRF-44 and CMRF-56). Young et al. (1988) Cell Immunol. 111:167; Van Voorhis et al. (1982) J. Exp. Med. 155:1172; Zhou et al. (1995) J. Immunol. 154:3821-3835; Fearnley et al. (1997) Blood 89:3708-3716; Mannering et al. (1988) J. Immunol. Met. 219:69-83; Hock et al. (1999) Tiss. Antigens 53:320-334; and Hock et al. (1994) Immunol. 83:573-581.

Functional CD1a⁺ DCs are typically generated ex vivo from monocytes and from CD34⁺ hematopoietic progenitor cells. Bender et al. (1996) J. Immunol. Met. 196:121-135; Pickl et al. (1996) J. Immunol. 157:3850-3859; Romani et al. (1994) J. Exp. Med. 180:83-93; Sallusto et al. (1994) J. Exp. Med. 179:1109-1118; Caux et al. (1992) Nature 360:258-261; Mackensen et al. (1995) Blood 86:2699-2707; Szabolcs et al. (1995) Immunol. 154:5851-5861; Herbst et al. (1996) Blood 88:2541-2548; de Wynter et al. (1998) Stem Cells 16:387-396; Strunk et al. (1996) Blood 87:1292-1302; and U.S. Pat. Nos. 6,010,905; and 6,004,807. It is not known if DCs generated in vitro from monocytes and hematopoietic progenitor cells retain or obtain all of the characteristics of in vivo DCs.

In addition, several attempts to generate mAb specific for human DC have failed, yielding only mAb that bind antigens expressed by both DC and other leukocytes. Human DC share a large number of immunogenic cell surface structures with other blood cells, including HLA molecules, CD18, CD29, CD31, CD43, CD44, CD45, CD54, and CD58. These antigens may dominate the immune response to injected DC to a level where B cells with specificity for DC-specific antigens are not at all or only very rarely represented among B cells that have the capability to fuse with myeloma cells.

Many investigators have tried to overcome this problem by injecting adult mice with non-DC and cyclophosphamide, in order to ablate B cells with specificity for shared antigens, or by injecting neonatal mice with non-DC, in order to tolerize B cells with specificity for shared antigens. O'Doherty et al. (1993) Adv. Exp. Med. Biol. 329:165-172; and Yamaguchi et al. (1995) J. Immunol. Met. 181:115-124.

A mAb designated CMRF44 has been used to monitor DCs in stem cell transplant patients. Fearnley et al. (1999) Blood 93:728-736. These CMRF44⁺ cells were proposed to be suitable for use in initiating, maintaining and directing immune responses. Fearnley et al. (1997). DCs have been isolated most often by using a combination of cell surface markers. For instance, U.S. Pat. No. 5,972,627 describes “hematopoietic cells enriched for human hematopoietic dendritic progenitor cells” as having “at least 80% expressing CD34, CD45RA, and CD10 but not CD19, CD2, CD3, CD4, CD8, CD20, CD14, CD15, CD16 CD56 and glycophorin.”

Isolation of DCs from blood relies on a multitude of immunophenotypic criteria, like the absence of a panel of leukocyte lineage (lin)-specific antigens (e.g. CD3, CD14, CD19 and CD56) and the presence of HLA-DR, CD4 or CD33. Romani et al. (1996) J. Immunol. Met. 196:137-151; Thomas et al. (1993); Thomas et al. (1994) J. Immunol. 153:4016-4028; O'Doherty et al. (1994) Immunol. 82:487-493; O'Doherty et al. (1993) J. Exp. Med. 178:1067-1076; Nijman et al. (1995) J. Exp. Med. 182:163-174; Ferbas et al. (1994) J. Immunol. 152:4649-4662; Heufler et al. (1996) Eur. J. Immunol. 26:659-668; Ito et al. (1999) J. Immunol. 163:1409-1419; Cella et al. (1999) Nature Med. 5:919-923; Robinson et al. (1999) Eur. J. Immunol. 29:2769-2778; Olweus et al. (1997) Proc. Natl. Acad. Sci. USA 94:12551-12556; Robert et al. (1999) J. Exp. Med. 189:627-636; and Kohrgruber et al. (1999) J. Inmunol. 163:3250-3259.

From analyses of DC isolated from non-cultured blood it became evident that blood DC are not a homogeneous cell population but a mixture of at least two populations. Thomas et al. (1994); O'Doherty et al. (1994); Ito et al. (1999); Cella et al. (1999); Robinson et al. (1999); Olweus et al. (1997); Kohrgruber et al. (1999); Strobl et al. (1998) J. Immunol. 161:740-748; and Rissoan et al. (1999). The first blood DC subpopulation is CD123^{bright} CD11c⁻DC, which possesses a plasmacytoid morphology and potent T cell stimulatory function. The second blood DC subpopulation is CD123dim CD11c^{bright}, which is rather monocytoid in appearance, expresses CD45RO and spontaneously develops into typical mature DCs even when cultured without any exogenous cytokines. Plasmacytoid CD₁₂₃^{bright} CD11c⁻DC display some features, like the expression of the pre-T cell receptor α chain, which indicate that they may arise from lymphoid precursors. Strobl et al. (1998); Rissoan et al. (1999); and Bruno et al. (1997) J. Exp. Med. 185:875-884. CD123^{bright} CD11c⁻DC display all the criteria of myeloid DCs. O'Doherty et al. (1994); and Ito et al. (1999). Robinson et al. (1999); Kohrgruber et al. (1999); and Strobl et al. (1998). DCs resembling plasmacytoid CD123^{bight}CD11c⁻ DC have been detected in the T cell-rich areas of lymphoid tissue and were initially erroneously designated plasmacytoid T cells or plasmacytoid monocytes due to their morphology and phenotype. Grouard et al. (1997) J. Exp. Med. 185:1101-1111; Lennert et al. (1975) Lancet 1:1031-1032; Lennert et al. (1984) in Leukocyte Typing. Human Leukocyte differentiation antigens detected by monoclonal antibodies. Bernard et al. eds. Springer-Verlag, Berlin; and Facchetti et al. (1998) Am. J. Pathol. 133:15-21. DCs resembling CD123^{dim}CD11c^{bright} blood DC have been found in the dark and light zone of germinal centers. Grouard (1996) Nature 384:364-367.

Additional immunologic functions are related to DCs such as differential induction of Th1 or Th2 responses, autoimmune reactions and allergies. Rissoan et al. (1999) Science 283:1183-1186; Hermans et al. (1998); and De Palma et al. (1999) J. Immunol. 162:1982-1987. Allergic responses, including those of allergic asthma and allergic rhinitis, are characterized by an early phase response, which occurs within seconds to minutes of allergen exposure and is characterized by infiltration of eosinophils into the site of allergen exposure. Specifically, during the early phase of the allergic response, activation of Th2-type lymphocytes stimulates the production of antigen-specific IgE antibodies, which in turn triggers the release of histamine and other mediators of inflammation from mast cells and basophils. During the late phase response, IL-4 and IL-5 production by CD4⁺ Th2 cells is elevated. These cytokines appear to play a significant role in recruiting eosinophils into the site of allergen exposure, where tissue damage and dysfunction result.

Currently, antigen immunotherapy for allergic disorders involves the subcutaneous injection of small, but gradually, increasing amounts, of antigen in a process called desensitization therapy. Antigen immunotherapy is merely palliative and, at present, not curative. Weber (1997) JAMA 278:1881-1887; Stevens (1998) Acta Clinica Beligica 53:66-72; and Canadian Society of Allergy and Clinical Immunology (1995) Can. Med. Assoc. J. 152:1413-1419.

Many patients who begin allergy therapy do not complete the regimen, and if injections are missed for over a week, the patient must begin the entire treatment regimen again. A variety of antigens have been identified and produced by recombinant means. For reviews, see Baldo et al. (1989) Allergy 44:81-97; Baldo (1991) Curr. Opin. Immunol. 3:841-850; Blaser (1994) Ther. Umsch 51:19-23; and Valenta et al. (1996) Adv. Exp. Med. Biol. 409:185-196.

Antigen immunotherapy treatments present the risk of inducing potentially lethal IgE-mediated anaphylaxis and do not address the cytokine-mediated events of the allergic late phase response. This therapy has been described as “having the potential for misadventure.” Weber (1997). Another significant problem with antigen immunotherapy is that the risk of adverse reactions, especially anaphylaxis, significantly reduces the dosage of antigen both with respect to the amount given per administration and the amount given over a period of time. Thus, traditional allergy immunotherapy is protracted and thus time-consuming, inconvenient, and expensive.

An alternative approach for treatment of IgE-associated disorders such as allergies involves administration of compounds that inhibit histamine release. Many such drugs are available as over-the-counter remedies. Other drugs include an anti-IgE binding antibody. However, a drawback of this approach is that it merely masks the symptoms, while not providing any kind of permanent cure or protection.

Increased levels of circulating IFN-α and of IFN-α-inducing factor (something like a complex of anti-DNA antibody and DNA) are found in SLE patients and correlate to disease activity. Furthermore, patients with non-autoimmune disorders treated with IFN-α frequently develop autoantibodies and occasionally SLE. Several papers from Ronnblom et al. ((1999) Clin. Exp. Immunol. 115: 196-202; (1999) J. Immunol. 163: 6306-6313; and (2000) J. Immunol. 165:3519-3526) show that IFN-α-inducing factors derived from patients induce secretion of IFN-α in PBMC from healthy donors and selectively activate natural IFN-α producing cells (NIPC=plasmacytoid DC).

### BRIEF DESCRIPTION OF THE INVENTION

The invention relates to methods of enriching for hematopoietic cell populations enriched in DCs and subsets thereof. It has now been found that a subset of DCs (BDCA-3) express CD141. CD141 has not previously been shown to be expressed on DCs. Thus, anti-CD141 antibodies and antigen binding fragments thereof are suitable for use in enriching, purifying and isolating DCs.

The invention further encompasses methods for obtaining compositions of hematopoietic cells enriched for DCs by separating, based on the expression of CD141, a mixture of human hematopoietic cells into a fraction wherein at least about 5%, at least about 10%, at least about 30%, at least about 50%, at least about 70%, or at least 80% of the cells in the fraction are BDCA-3⁺.

The invention further encompasses methods for isolating a substantially pure subset of DCs by a) obtaining a mixture of human hematopoietic cells; and b) substantially isolating cells from the mixture specifically recognized by an antigen-binding fragment specific for CD141.

The invention further encompasses methods of producing DC cytokines by isolating a substantially pure population or subpopulation of DCs with an antigen-binding fragment specific for CD141; and isolating cytokines from the cells or cellular products or supernatants.

The invention further encompasses methods of modulating DC cytokine production by isolating a substantially pure population or subpopulation of DCs with an antigen-binding fragment specific for CD141; and treating the cells with agents that modulate DC cytokine production.

The invention further encompasses a method of inhibiting an interaction of a DC with a T cell by contacting a composition containing DC and T cells with an effective amount of an agent that inhibits the interaction of BDCA-3 with the T cell by interfering specifically with CD141.

The invention further encompasses a method of treating inflammation by administering to a subject in need thereof an amount of an agent that inhibits the interaction of BDCA-3 with the T cell by interfering specifically with CD141 effective to reduce inflammation in the subject.

### BRIEF DESCRIPTION OF THE DRAWINGS

FIG. 1 is a series (A-H) showing the results of FACS analysis of various populations of cells. A and B depict properties and staining with propidium iodide (PI; 1 μg/ml); C-E show staining of unstimulated HD-MY-Z cells; F-H show PMA/Ionomycin stimulated cells.

FIG. 2 shows a stained gel containing three different protein bands. Protein 1 (at about 100 kD) corresponds to the molecular weight of BDCA-3.

FIGS. 3A and 3B show the further characterization the three marked proteins sequenced with Maldi-TOF at Protagen AG. Protein 1 is thrombomodulin (TM, SEQ ID NO: 4 in FIG. 3B); protein 2 is most likely a BSA contaminant (SEQ ID NO: 2); and protein 3 is a dimer of thrombomodulin. FIG. 3A (SEQ ID NO: 1) shows an earlier sequence reported for TM.

FIG. 4 is a series (A-H) showing the results of FACS analysis of various populations of cells. A-D show staining of control cells; and E-H show staining of hTM-pSR1neo transfected COS-7 cells. Anti-BDCA-3 antibodies AD5-5E8 and AD5-14H12 as well as the CD141-specific mAb specifically bind to TM-transfected cells (F and G) whereas they do not stain the control cells (B and C). Isotype control staining with CD19.PE is negative on both cells (A and E).

FIG. 5 shows that binding of 1A4 is not blocked by the anti-BDCA-3 antibodies, i.e. 1A4 recognizes a different epitope than anti-BDCA-3 antibodies AD5-5E8 and -14H12. A shows CD141.PE staining of HD-MY-Z with clone 1A4; B shows CD141.PE staining of HD-MY-Z with clone 1A4 after pre-incubation with 200 μg/ml AD5-14H12; and C shows CD141.PE staining of HD-MY-Z with clone 1A4 after pre-incubation with 200 μg/ml AD5-5E8.

FIG. 6 is a series (A-J) showing the results of FACS analysis of various populations of cells. Dead cells and cell debris were excluded according to their scatter properties and staining with propidium iodide (PI; 1 μg/ml) (A and B) and CD14+ monocytes were excluded according to staining with CD14.PerCP (B). Staining of cells with CD141.PE versus AD5-14H12 before (C) and after (G) MACS separation. Counterstaining with CD11c.FITC versus either AD5-14H12 before (E) and after (I) MACS separation or CD141.PE before (D) and after (H) MACS separation.

FIG. 7 shows the isolation of BDCA-3⁺ blood dendritic cells from PBMC using the magnetic beads conjugated to monoclonal antibody AD5-5E8.

### DETAILED DESCRIPTION OF THE INVENTION

The practice of the present invention will employ, unless otherwise indicated, conventional techniques of molecular biology (including recombinant techniques), microbiology, cell biology, biochemistry, nucleic acid chemistry, and immunology, which are within the skill of the art. Such techniques are explained fully in the literature, such as, Current Protocols in Immunology (J. E. Coligan et al., eds., 1999, including supplements through 2001); Current Protocols in Molecular Biology (F. M. Ausubel et al., eds., 1987, including supplements through 2001); Molecular Cloning: A Laboratory Manual, third edition (Sambrook and Russel, 2001); The Immunoassay Handbook (D. Wild, ed., Stockton Press NY, 1994); Bioconjugate Techniques (Greg T. Hermanson, ed., Academic Press, 1996); Methods of Immunological Analysis (R. Masseyeff, W. H. Albert, and N. A. Staines, eds., Weinheim: VCH Verlags gesellschaft mbH, 1993), and Harlow and Lane (1999) Using Antibodies: A Laboratory Manual Cold Spring Harbor Laboratory Press, Cold Spring Harbor, N.Y.

The invention relates to methods of analyzing or enriching for cell populations enriched in DCs and subsets thereof. Compositions enriched for the DCs and populations of cells obtained therefrom are also provided by the invention. Methods and compositions for modified cells are also provided. Compositions of modified cells, including genetically modified cells are also provided. Methods of use of the cells both modified and non-modified are provided. Antigen-binding fragments and the antigens recognized thereby are also provided.

In particular, the invention relates to the subset of dendritic cells that express the BDCA-3 antigen. This antigen has now been identified as CD141 or thrombomodulin (TM). Expression of the BDCA-3 antigen is described in PCT publication WO 01/36487, copending U.S. patent application no. 09/714,712, and Dzionek et al., 2000, J. Immunol. 165:6037-46 (each incorporated by reference in their entirety for all purposes.) Expression was detected in a small population of CD1c⁻CD11c⁺CD123⁻DC in non-cultured human blood. This population is referred to herein as BDCA-3 dendritic cells. The cell surface phenotype of BDCA-3 dendritic cells is CD₁₄₁^{bright} CD11c⁺, CD14⁻, CD1c⁻, CD123⁻ (see Dzionek et al., supra).

With respect to phenotype, morphology, endocytic capacity, and maturation requirements, this DC population is quite similar to the CD1c⁺CD11c^{bright}CD123^{dim} DC population. However, apart from BDCA-3 and CD1c expression themselves, the immunophenotypic analysis has revealed some striking differences: in contrast to CD1c⁺ BDC, BDCA-3⁺ BDC do not express the Fc receptors CD32, CD64 and FcεRI, and they do not express CD2. The lack of Fc receptor expression indicates that BDCA-3⁺ BDC, unlike CD1c⁺ BDC do not have the capability of Ig-mediated antigen uptake. Fanger et al. (1996) J. Immunol. 157:541-548; Fanger et al. (1997) J. Immunol. 158:3090-3098; and Maurer et al. (1996) J. Immunol. 157:607-616. As shown herein and in WO 01/36487, BDCA-3 is a 100 kD protein.

There is evidence that CD1c⁺CD11c^{bright} DC, in contrast to CD1c⁻CD11c⁺ DC, have the capacity to acquire Langerhans cell characteristics (expression of Lag antigen, E-cadherin and Langerin, and presence of Birbeck granules) when cultured with GM-CSF, IL-4 and TGF-β1. If BDCA-3⁺ DC and CD1c⁺ DC represent maturational stages of the same cell type, this would indicate that BDCA-3⁺ DC have either already lost or not yet acquired the capacity to differentiate into Langerhans cells.

Staining of CD1c⁺ DC for CD1c, CD2 and CD14 revealed that a minor proportion of DC expresses CD14 to a variable degree and that the level of CD1c as well as CD2 expression on these cells is inversely proportional to the level of CD14 expression. This observation is in accordance with a linear differentiation model, where CD1c⁺CD2⁺CD11c^{bright}CD14⁻ DC are the progeny of CD14⁺CD1c⁻CD2⁻ monocytes rather than the progeny of a common precursor of both cell types. This concept finds further support by the observation that a considerable proportion of CD14⁺ monocytes already express very low levels of CD2 and have the capacity to rapidly differentiate into mature DC with typical dendritic morphology and potent T cell stimulatory function when cultured with GM-CSF and IL-4. Crawford et al. (1999) J. Immunol. 163:5920-5928.

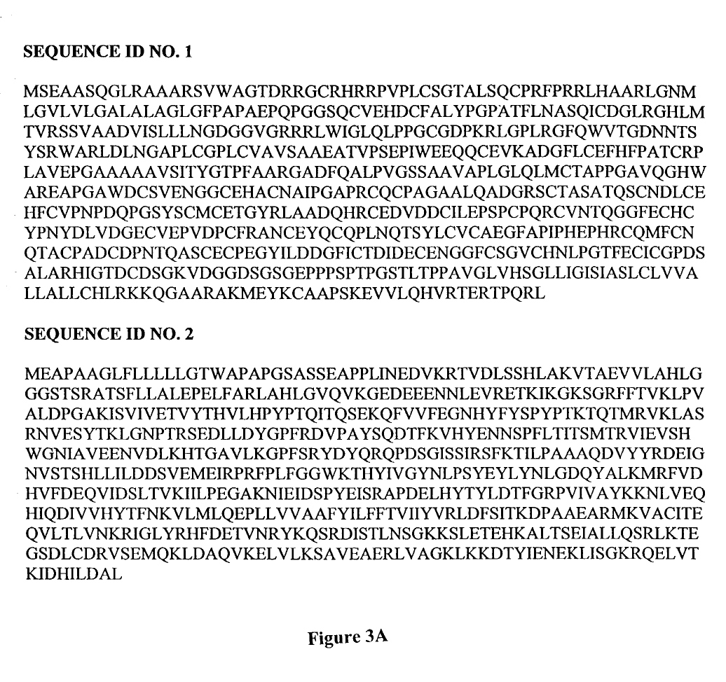

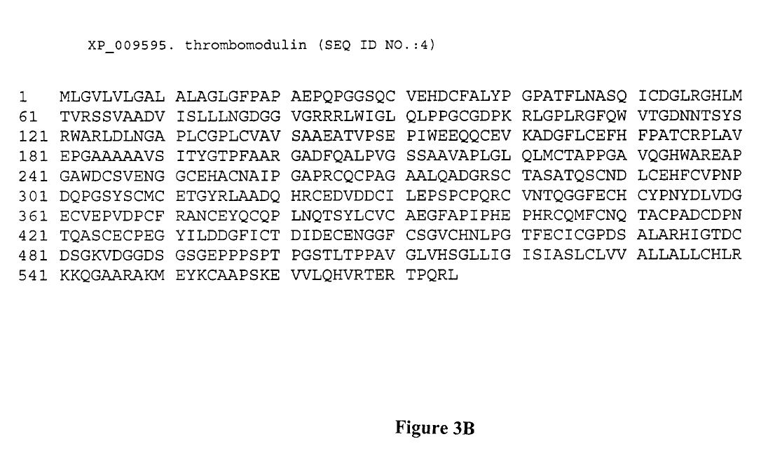

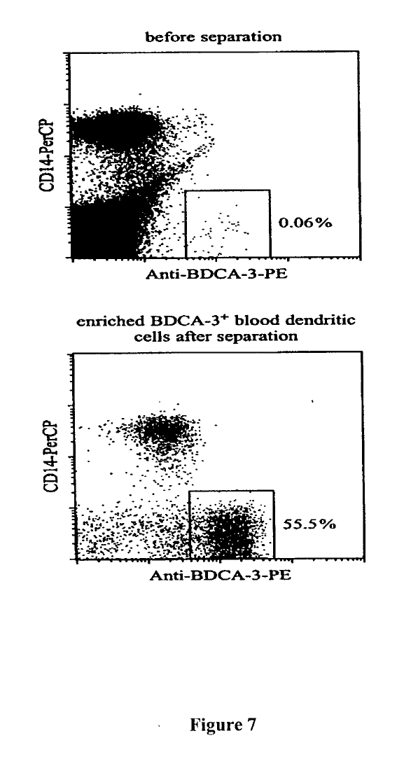

CD141 has not previously been shown to be expressed on DCs. Thus, anti-CD141 antibodies and antigen binding fragments thereof are suitable for use in enriching, purifying and isolating DCs. CD141, also called thrombomodulin (TM), is a vascular endothelial cell surface receptor critical for hemostasis. CD141 initiates the protein C anticoagulant pathway. The sequence of CD141 is found at in FIG. 7 (SEQ ID NO.:3) and at GenBank accession no. NCBI XP_{—}009595) (showing a 575 residue sequence including a signal peptide of about 21 amino acids, an amino terminal ligand-binding domain of about 223 amino acids, an epidermal growth factor (EGF) homology region of 236 amino acids, a serine/threonine-rich segment of 34 amino acids, a membrane-spanning domain of 23 amino acids, and a cytoplasmic tail of 38 amino acids. A related earlier sequence reported for TM is provided in FIG. 3 (SEQ ID NO.:1). Upon binding to CD141, thrombin loses its procoagulant functions and the CD141/thrombin complex is able to efficiently convert protein C to its activated form. CD141 is reported to by expressed on endothelial cells, keratinocytes, megakaryocytes, platelets, monocytes, neutrophils, smooth muscle cells and synovial lining cells. Chu et al. (2000) Nature 404:741-743; Banner (2000) Nature 404:449-450; and Conway et al. (1997) Blood 89:652-661.

The invention further encompasses methods for obtaining compositions of hematopoietic cells enriched for DCs by separating or enriching for dendritic cells from other cells in a population (e.g., a mixture of human hematopoietic cells) based on the expression of CD141. In embodiments, the enrichment of dendritic cells is at least 10-fold over the starting material, and sometimes is at least about 20-fold, at least about 50-fold, at least about 100-fold, at least about 500-fold or at least about 1000-fold. In embodiments, enrichment or separation results in a composition in which at least about 3%, at least about 5%, at least about 10%, at least about 30% of the cells, sometimes at least about 50% of the cells, and sometimes at least about 80% or more of the cells in the fraction are BDCA-3⁺. In embodiments, the enrichment or separation results in a composition in which at least about 3%, at least about 5%, at least about 10%, at least about 30%, at least about 50%, at least about 70%, and sometimes at least about 80% or more of the cells in the fraction are BDCA-3⁺. In some embodiment, the composition is a substantially pure population of BDCA-3⁺ dendritic cells, i.e., in which at least about 40% of the cells, generally at least about 50% of the cells, and sometimes at least about 80% or more of the cells in the fraction are BDCA-3⁺. The dendritic cell-enriched cell populations can be used for any purpose for which dendritic cells are suited, and, for example, can be cultured, manipulated or admininstered to subjects. Thus, in an aspect, the invention provides dendritic cells and dendritic cell-enriched composition obtained by the methods disclosed herein, and progeny of such cells (e.g., obtained by culturing cells obtained by the enrichment method). Exemplary compositions have at least about 30% dendritic cells (e.g., at least about 30% CD1c⁻, CD11c⁺, CD123⁻ dendritic cells) or at least about 80% dendritic cells.

The invention further encompasses methods for isolating an enriched, purified or substantially pure subset of DCs by a) obtaining a mixture of human hematopoietic cells; and b) substantially isolating cells from the mixture specifically recognized by an antigen-binding fragment specific for CD141.

The invention further encompasses methods of producing DC cytokines by isolating a substantially pure population or subpopulation of DCs with an antigen-binding fragment specific for CD141; and isolating cytokines from the cells or cellular products or supernatants.

The invention further encompasses methods of modulating DC cytokine production by isolating a substantially pure population or subpopulation of DCs with an antigen-binding fragment specific for CD141; and treating the cells with agents that modulate DC cytokine production.

The invention further encompasses method of inhibiting an interaction of a dendritic cell with a ligand that binds CD141 on the dendritic cell surface by contacting the dendritic cell with a CD141 binding fragment or reducing the expression of CD141 on the dendritic cell surface. For example, a method of inhibiting an interaction of a DC with a T cell by contacting a composition containing DC and T cells with an effective amount of an agent that inhibits the interaction of BDCA-3 with the T cell by interfering specifically with CD141 is provided. As used herein, “interfering with CD141” includes, without limitation, binding an agent (e.g., CD141 binding fragment) to CD141 on the cell surface, so that the interaction of CD141 and ligands is reduced and specifically reducing expression of CD141 on the cell surface (for example by using TM antisense RNA to reduce expression of CD141 protein). Other agents that reduce thrombomodulin expression include interleukin-1, tumour necrosis factor and endotoxin. The invention further encompasses a method of treating inflammation by administering to a subject in need thereof an amount of an agent that inhibits the interaction of BDCA-3 with the T cell by interfering specifically with CD141 effective to reduce inflammation in the subject. In an embodiment, the invention provides a method of inhibiting an interaction of a dendritic cell and a T cell by contacting the dendritic cell with a CD141 binding fragment or reducing the expression of CD141 on the dendritic cell surface.

In an embodiment, the invention provides a method of enriching for dendritic cells from a mixture of cells by contacting the mixture of cells with an antigen-binding fragment specific for CD141 and selecting the cells that are CD141⁺.

The mixture of cells (e.g., unpurified source of dendritic cells) may be any known in the art, such as the bone marrow, fetal, neonate or adult or other hematopoietic cell source, e.g., fetal liver, peripheral blood or umbilical cord blood tonsil, lymph node, nasal membrane, spleen, skin, airway epithelia, lung, liver gut, Peyers patches, etc. Often the mixture of cells is blood (e.g. peripheral blood or umbilical cord blood) or a blood fraction (e.g., perpheral blood mononuclear cells or perpheral blood lymphocytes). DCs can also be isolated from populations of cultured cells such as DCs derived from progenitor cells, such as cultured cells from blood. Usually, the cells are human. However, the methods of the invention can be carried out using nonhuman cells and antibodies that bind nonhuman homologs of human CD141. In one embodiment tissue (e.g., blood) or a tissue fraction (e.g., blood fraction) is obtained from a subject treated with an agent that increases the number of dendritic cells resident in peripheral blood. Such agents are known and include, for example, Flt3-Ligand and homologs or analogs, G-CSF and homologs or analogs, and agents that induce expression in the subject of proteins (e.g., endogenous proteins) that result in mobilization.

Suitable antigen-binding fragment specific for CD141 are well known in the art and are described below. Examples of commercially available anti-CD141 monoclonal antibodies include: clone 1A4 (described in HLDA workshop No.: VI E013 and available from BD Pharmingen; see Examples, infra); clone QBEND/40 (available from Serotec); clone 1009 (available from Novocastra and Dako); clone 15C8 (available from Novocastra). It will be within the ability of the ordinarily skilled practicioner to identify and/or prepare other CD141 binding fragments based on the guidance disclosed herein. It will be apparent, for example, that for cell staining and selection, anti-CD141 antibodies used in the invention, for example, will bind to the extracellular portion of CD141. In a specific embodiment, the monoclonal antibodies designated AD5-5E8 and AD5-14H12 are not used, i.e., are excluded from the invention. For many applications, the binding fragment (e.g. anti-CD141 monoclonal antibody) is detectably labeled, e.g., as described herein. Often the binding fragment is magnetically or is labeled with a fluorescent tag (e.g., FITC).

Contacting can occur in any of a number of ways, depending on the nature of the starting material and ultimate goal. For example, the CD141 binding fragment can be added to whole blood, a blood fraction, cultured cells, a tissue lysate, or the like.

Procedures for enrichment or separation of cells (e.g., cells labeled with a anti-CD141 antibody) include, but are not limited to, density gradient centrifugation; rosetting; coupling to particles that modify cell density; magnetic separation with antibody-coated magnetic beads or antibody-coated fero fluids (nonoparticles); flow cytometry, affinity chromatography; cytotoxic agents joined to or used in conjunction with an anti-CD141 mAb, including, but not limited to, complement and cytotoxins; and panning with antibody attached to a solid matrix, e.g. plate, elutriation or any other convenient technique.

In one embodiment of the invention, the anti-CD141 antibodies are coupled to a magnetic reagent, such as a superparamagnetic microparticle (microparticle). Herein incorporated by reference, Molday (U.S. Pat. No. 4,452,773) describes the preparation of magnetic iron-dextran microparticles and provides a summary describing the various means of preparing particles suitable for attachment to biological materials. A description of polymeric coatings for magnetic particles used in high gradient magnetic separation (HGMS) methods are found in DE 3720844 (Miltenyi) and U.S. Pat. No. 5,385,707. Methods to prepare superparamagnetic particles are described in U.S. Pat. No. 4,770,183. See also See, e.g., U.S. Pat. Nos. 5,779,892 (Magnetic separator with magnetic compensated release mechanism for separating biological material); 5,711,871 (Magnetic separation apparatus); 5,411,863 (Methods and materials for improved high gradient magnetic separation of biological materials). The microparticles will usually be less than about 100 nm in diameter, and usually will be greater than about 10 nm in diameter. The exact method for coupling is not critical to the practice of the invention, and a number of alternatives are known in the art. Direct coupling attaches the separation antibodies to the particles. Indirect coupling can be accomplished by several methods. The antibodies may be coupled to one member of a high affinity binding system, e.g. biotin, and the particles attached to the other member, e.g. avidin. One may also use second stage antibodies that recognize species-specific epitopes of the antibodies, e.g. anti-mouse Ig, anti-rat Ig, etc. Indirect coupling methods allow the use of a single magnetically coupled entity, e.g. antibody, avidin, etc., with a variety of separation antibodies.

A suspension of cells is applied to a separation device. Exemplary magnetic separation devices are described in WO/90/07380, PCT/US96/00953 and EP 438,520, herein incorporated by reference. In a preferred embodiment, an improvement is provided by the use of a high gradient magnetic matrix of closely packed ferromagnetic spheres in place of the prior art matrix of steel wool, wires, etc. The spheres will be usually at least about 200 μm in diameter and not more than about 1000 micrometers in diameter, more usually at least about 250 μm in diameter and not more than about 300 micrometers in diameter. For optimum performance it is preferred that the composition of spheres be generally homogeneous in size, usually varying not more than about 15% from the average size. The spheres are composed of a ferromagnetic material (e.g. iron, steel, etc.), which may be coated with an impermeable coating to prevent the contact of cells with metal. By impermeable coating it is meant a polymeric coating which contains substantially less than 30% water by weight, which does not permit the passage of ions, and which is formed on the sphere as a result of passive application, cross-linking or polymerization of a relatively hydrophobic polymer or co-polymer. Suitable polymers include polystyrenes, polyacrylamides, polyetherurethanes, polysulfones, fluorinated or chlorinated polymers such as polyvinyl chloride, polyethylenes and polypropylenes, polycarbonates and polyesters, etc. The matrix of spheres should have adequate surface area to create sufficient magnetic field gradients in the separation device to permit efficient retention of magnetically labeled cells. The volume necessary for a given separation may be empirically determined, and will vary with the cell size, antigen density on the cell surface, antibody affinity, etc. The flow rate will be determined by the size of the column, but will generally not require a cannula or valve to regulate the flow.

The labeled cells are retained in the magnetic separation device in the presence of a magnetic field, usually at least about 100 mT, more usually at least about 500 mT, usually not more than about 2 T, more usually not more than about 1 T. The source of the magnetic field may be a permanent or electromagnet. After the initial binding, the device may be washed with any suitable physiological buffer to remove unbound cells. Preferably, the cells are collected in a medium comprising 2% serum, such as fetal calf serum (FCS) or, human serum albumin (HSA) or any other suitable, preferably sterile, isotonic medium. For physiologic indications, HSA is preferred.

The unbound cells contained in the eluate are collected as the eluate passes through the column. The bound cells, e.g., containing dendritic cells, are released by removing the magnetic field, and eluting in a suitable buffer. The cells may be collected in any appropriate medium. Various media are commercially available and may be used according to the nature of the cells, including dMEM, HBSS, dPBS, RPMI, PBS-EDTA, PBS, Iscove's medium, etc., frequently supplemented with fetal calf serum, BSA, HSA, etc.

In many cases a single separation step will provide sufficient enrichment of dendritic cells. Where greater purity is desired, additional separation steps may be performed. The eluted, magnetic fraction may be passed over a second magnetic column to reduce the number of non-specifically bound cells. Alternatively, a multiparameter separation may be performed, by depleting hematopoietic cells from the sample.

Techniques providing accurate separation and analysis also include, but are not limited to, flow cytometry, which can have varying degrees of sophistication, e.g., a plurality of color channels, low angle and obtuse light scattering detecting channels, impedance channels, etc.

Any number of techniques can be employed to separate the dendritic cells from other cells. For instance, negative selection methods for depletion of nondendritic cell types from the mixture. The cells can be selected against dead cells, by employing dyes associated with dead cells such as propidium iodide (PI). mAbs are particularly useful for identifying markers associated with particular cell lineages and/or stages of differentiation for both positive and negative selections. If desired, a large proportion of terminally differentiated cells can be initially removed using a relatively crude negative separation. For example, magnetic bead separations can be used initially to remove large numbers of irrelevant cells. At least about 50%, sometimes at least about 70% or 80% of the total cells will be removed prior to isolation of DCs. In an embodiment, the DCs are directly isolated from the cell source by positive selection

For greatest purity, the isolation or enrichment of CD141⁺ dendritic cells can additionally include steps of removing other cells (e.g., including CD141⁺ cells other than dendritic cells or other than BDCA-3 dendritic cells) from the population. That is, the mixture can be depleted of non-dendritic cells, e.g., non BDCA-3 dendritic cells. The other cells can be removed from the population by any of a number of methods known in the art, and can be removed prior to or after initial enrichment of dendritic cells using CD141 binding fragments. Methods for removing other cells include, for example, gradient centrifugation, or removal based on labeling cell surface antigens not found on BDCA-3 dendritic cells. For example, anti-CD14 can be used for detection of co-isolated monocytes (see, e.g., Examples), anti-CD15 can be used for removal of CD15⁺ neutrophils; anti-CD1c can be used for the detection of co-isolated CD1c (BDCA-1⁺) myoloid dendritic cells; and the like. In addition, nondendritic cells can be distinguished from dendritic cells on the basis of morphology, behavior in culture (e.g., adherence to a substrate) or the like.

When dendritic cells are isolated from cell populations that contain other blood cell types that express CD141, it will often be useful to titrate the concentration of CD141 antibody concentration for labelling so that CD141^{bright} cells are labelled while CD141^{dim} cells still appear negative. Such a titration is well known within the art.

In a related aspect, the invention provides a method of depleting dendritic cells (e.g., CD1c−CD11c+CD123− cells) from a population of peripheral blood mononuclear cells by contacting said population with a CD141 antigen-binding fragment and removing cells that are CD141+.

Dendritic cells and cell compositions of the invention have a variety of uses, including use as vaccine adjuvants. See, e.g., Kumamoto et al., J Dermatol. 2001 28:658-62; Mumper et al., Mol Biotechnol. 2001 19:79-95; Bubenik et al., Int J Oncol. 2001 8:475-8; Somasse et al,Adv. Exp. Med. Biol. 329:299-303, 1993. Thus, in one aspect, the invention provides a method of generating an antigen-specific immune response specific by administering a dendritic cell that presents the antigen as an MHC protein-antigen complex. In an embodiment, the immune response is a T cell response.

Genetic modification of the cells (e.g., dendritic cells) can be accomplished at any point during their maintenance by transducing a substantially homogeneous cell composition with a recombinant DNA construct, transfected with RNA, cell fusion, loading with antigens and various methods known in the art and/or described herein.

For modification of the cells, a retroviral vector can be employed, however any other suitable vector, delivery system or cellular modification can be used. These include, e.g., adenovirus, adeno-associated virus, artificial chromosomes, derived from yeast and RNA derived from an antigen source such as a tumor. The genetic modification, if any, need not be permanent as mature DCs have a limited lifetime. Genetic approaches are used to express foreign (tumor, viral, parasitic, etc.) antigens or autoantigens in DCs in order to induce immunity or tolerance. The longevity of the modification can also be controlled by suicide genes to limit therapy (as with T cells).

Methods of transduction include any known in the art including, without limitation, direct co-culture of the cells with producer cells, e.g., by the method described by Bregni et al. (1992) Blood 80:1418-1422, or culturing with viral supernatant alone with or without appropriate growth factors and polycations, e.g., by the method described by Xu et al. (1994) Exp. Hemat. 22:223-230; and Hughes et al. (1992) J. Clin. Invest. 89:1817.

Upon reintroduction of the modified cells expressing or loaded with an antigen so as to present the antigen, into the host, T cells are activated, anergized or deleted and are specifically directed against the antigen. Generally, suitable antigens include those expressed by virally infected cells, or cancer cells, bacteria, yeast, protozoan, autoantigens (tolerogens) and allergens. More specifically, suitable antigens include, but are not limited to, viral proteins, proteins of cancer cells, tissue-specific proteins or tolerogenic proteins. “Induction” of T cells can include inactivation of antigen-specific T cells such as by deletion or anergy. Inactivation is particularly useful to establish or reestablish tolerance such as in organ transplantation and autoimmune disorders respectively. The modified DCs can be administered by any method known in the art including, but not limited to, intravenously, subcutaneously, intranodally and directly to the thymus. Preferably, administration is intravenous (IV).

Often, cell immunotherapy involves removal of bone marrow leukopheresis harvests or other source of cells from a human host, isolating the cells from the source. Meanwhile, the host may be treated to partially, substantially or completely ablate native hematopoietic capability if hematopoietic stem cell transplantation is to occur. The isolated cells can be modified during this period of time, so as to provide for cells having the desired modification. In the case of complete hematopoietic ablation, stem cell augmentation will also be required. The cells or modified cells can then be restored to the host to provide for the new capability. The methods of cell removal, host ablation and stem/progenitor cell repopulation are known in the art.

The modified cells can be administered in any physiologically acceptable vehicle, normally intravascularly, intranodal and subcutaneously. Usually, at least 1×10⁵ cells will be administered, preferably 1×10⁶ or more. The cells can be introduced by injection, catheter, or the like. If desired, factors can also be included, including, but not limited to, interleukins, e.g. IL-2, IL-3, IL-4, IL-12, and flt-Ligand, as well as the other interleukins, the colony stimulating factors, such as G-, M- and GM-CSF, interferons, e.g. γ-interferon.

Other uses of dendritic cells and populations include screening assays for agents that affect dendritic cell function (e.g., cytokine secretion profiles) and the like, phenotypic, functional, biochemical or molecular analyses of dendritic cells; enumeration of myeloid BDCA-3+ blood dendrific cells e.g. in the course of disease or after hematopoietic stem cell transplantation.

The dendritic cells can be treated in a number of ways, as described herein. The treatments can take place prior to the enrichment step (e.g., by contacting a cell mixture, e.g., blood, with an agent) or after an enrichment step (e.g., by contacting an enriched population of dendritic cells with an agent). The contacting can be in vitro or in vivo. Examples of treatments inlcude treatment with agents that downregulate thrombomodulin expression (interleukin-1, tumour necrosis factor and endotoxin) or upregulate TM (e.g., IL-3, or agents that increase cyclic AMP such as forskolin). Other methods for affecting or activating dendritic cells (e.g., administration of phorbol ester (e.g. PMA), ionophores (e.g. Ionomycin), LPS, CpG, and/or cytokines, or CD40 ligation) are well known.

The term “polypeptide”, “peptide” and “protein” are used interchangeably herein to refer to polymers of amino acid residues of any length. The polymer can be linear or branched, it can comprise modified amino acid residues or amino acid analogs, and it can be interrupted by chemical moieties other than amino acid residues. The terms also encompass an amino acid polymer that has been modified naturally or by intervention; including, but not limited to, disulfide bond formation, glycosylation, lipidation, acetylation, phosphorylation, or any other manipulation or modification, such as conjugation with a labeling or bioactive component. Unless stated or implied otherwise, the term antigen-binding fragment includes any polypeptide monomer or polymer with immunologic specificity, including the intact antibody, and smaller and larger functionally equivalent polypeptides, as described herein.

1. Antigen-binding Fragments and Compositions Thereof

The use of CD141 mAb provides a convenient and efficient way to rapidly detect, enumerate and isolate DC populations from PBMC, leukapheresis material, whole blood, tonsil, etc., without apparent functional perturbation. This is a valuable aid for their further functional and molecular characterization and can be useful in elucidating their interrelationships. Furthermore, the ability to easily isolate DC populations to homogeneity greatly facilitates their clinical use. The antigen-binding fragments are also useful in detecting, enumerating and/or isolating DCs from blood and other tissues, both non-hematopoietic tissues (including, without limitation, airway epithelia, skin, gut, lung, and liver) and hematopoietic tissues (including, without limitation, tonsil, spleen, lymph node and thymus).

Hybridomas are useful in producing antibodies are other cells expressing antigen-binding fragments. As seen from the Examples provided herein, multiple types of mAbs can be produced which specifically recognize this antigen. As also seen from the results presented herein, the antigen-binding fragments need not recognize the same epitope on the same antigen.

The term “antigen-binding fragment” includes any moiety that binds preferentially to a DC or a sub-population thereof. Suitable moieties include, without limitation, oligonucleotides known as aptomers that bind to desired target molecules (Hermann and Pantel (2000) Science 289:820-825), carbohydrates, lectins, Ig fragments as Fab, F(ab′)₂, Fab′, scFv (both monomer and polymeric forms) and isolated H and L chains. An antigen-binding fragment retains specificity of the intact Ig, although avidity and/or affinity can be altered.

Certain compounds, compositions and methods described herein relate generally to antibodies and derivatives thereof which having provided the antigenic determinants herein, can be generated routinely by standard immunochemical techniques. These include, but are not limited to, antigen-binding fragments coupled to another compound, e.g. by chemical conjugation, or associated with by mixing with an excipient or an adjuvant. Specific conjugation partners and methods of making them are described herein and known in the art.

Antigen-binding fragments (also encompassing “derivatives” thereof) are typically generated by genetic engineering, although they can be obtained alternatively by other methods and combinations of methods. This classification includes, but is not limited to, engineered peptide fragments and fusion peptides. Preferred compounds include polypeptide fragments containing the anti-DC CDRs, antibody fusion proteins containing cytokine effector components, antibody fusion proteins containing adjuvants or drugs, antibody fusion proteins containing tumor cell-derived antigens, viral antigens, bacterial antigens, parasite antigens, yeast antigens, autoantigens or antigenic peptides (T cell epitopes) derived therefrom, and, single chain V region proteins. Antigen-binding fragments are considered to be of human origin if they are isolated from a human source, and used directly or cloned and expressed in other cell types and derivatives thereof or whole human chromosomes or portions thereof (such as mice with human chromosomes encoding V_{H}, D_{H}, J_{H}, V_{L}, JL_{L}, C_{H}, C_{L} gene segments).

A “fusion polypeptide” is a polypeptide comprising contiguous peptide regions in a different position than would be found in nature. The regions can normally exist in separate proteins and are brought together in the fusion polypeptide; they can normally exist in the same protein but are placed in a new arrangement in the fusion polypeptide; or they can be synthetically arranged. For instance, the invention encompasses recombinant proteins (and the polynucleotides encoding the proteins or complementary thereto) that are comprised of a functional portion of an antigen-binding fragment and another peptide such as a toxin. Methods of making these fusion proteins are known in the art and are described for instance in WO93/07286.

A “functionally equivalent fragment” of a polypeptide varies from the native sequence by any combination of additions, deletions, or substitutions while preserving at least one functional property of the fragment relevant to the context in which it is being used.

The antigen-binding fragments are useful in palliating the clinical conditions related to immunologic disorders. The invention further comprises polypeptide derivatives of the antigen-binding fragments and methods for using these compositions in diagnosis, treatment, and manufacture of novel reagents.

Antigen-binding fragments can also be conjugated to a chemically functional moiety. Typically, the moiety is a label capable of producing a detectable signal. These conjugated antigen-binding fragments are useful, for example, in detection systems such as quantitation of DCs in various tissues, in various diseases, after stem cell transplantation, and after immunoablative therapy like chemotherapy and radiation, and imaging of DCs for instance in following chemotherapy or autoimmune therapy. Such labels are known in the art and include, but are not limited to, radioisotopes, enzymes, fluorescent compounds; chemiluminescent compounds, bioluminescent compounds, substrate cofactors and inhibitors and magnetic particles. For examples of patents teaching the use of such labels, see, for instance U.S. Pat. Nos. 3,817,837; 3,850,752; 3,939,350; 3,996,345; 4,275,149; 4,277,437; and 4,366,241. The moieties can be covalently linked, recombinantly linked, or conjugated (covalently or non-covalently) through a secondary reagent, such as a second antibody, protein A, or a biotin-avidin complex.

Other functional moieties include, without limitation, signal peptides, agents that enhance immunologic reactivity, agents that facilitate coupling to a solid support, vaccine carriers, bioresponse modifiers, paramagnetic labels and drugs. Signal peptides include prokaryotic and eukaryotic forms. Agents that enhance immunologic reactivity include, but are not limited to, bacterial superantigens and adjuvants. Agents that facilitate coupling to a solid support include, but are not limited to, biotin, avidin or derivatives thereof. Immunogen carriers include, but are not limited to, any physiologically acceptable buffer. Bioresponse modifiers include, but are not limited to, cytokines, particularly tumor necrosis factor (TNF), IL-2, interleukin-4 (IL-4), GM-CSF; IL-10, IL-12, TGF-β and certain interferons, and chemokines (MIP-3β, SDF-1, Lymphotactin, DC-CK1, Eotaxins, IP-10, TARC, Rantes, MIP-1x, MIP-1B, SLC, 1-TAC, MIG, MDC, MCP-1, TCA-3, MCP-2,- 3, -1. See also, U.S. Pat. No. 5,750,119; and WO patent publications: 96/10411; 98/34641; 98/23735; 98/34642; 97/10000; 97/10001; and 97/06821. Such, chemokines may be useful to attract other cells such as T cells.

A “signal peptide” or “leader sequence” is a short amino acid sequence that directs a newly synthesized protein through a cellular membrane, usually the endoplasmic reticulum (ER) in eukaryotic cells, and either the inner membrane or both inner and outer membranes of bacteria. Signal peptides are typically at the N-terminus of a polypeptide and are removed enzymatically between biosynthesis and secretion of the polypeptide from the cell or through the membrane of the ER. Thus, the signal peptide is not present in the secreted protein but is present only during protein production.

Immunotoxins, including single chain conjugates, can be produced by recombinant means. Production of various immunotoxins is well known in the art, and methods can be found, for example, in “Monoclonal Antibody-toxin Conjugates: Aiming the Magic Bullet,” Thorpe et al. (1982) Monoclonal Antibodies in Clinical Medicine, Academic Press, pp. 168-190; Vitatta (1987) Science 238:1098-1104; and Winter and Milstein (1991) Nature 349:293-299. Suitable toxins include, but are not limited to, ricin, radionuclides, pokeweed antiviral protein, Pseudomonas exotoxin A, diphtheria toxin, ricin A chain, fungal toxins such as fungal ribosome inactivating proteins such as gelonin, restrictocin and phospholipase enzymes. See, generally, Olsnes et al. (1981) Pharmac. Ther. 15:355-381; and “Monoclonal Antibodies for Cancer Detection and Therapy,” eds. Baldwin and Byers, pp. 159-179, 224-266, Academic Press (1985).

The chemically functional moieties can be made recombinantly for instance by creating a fusion gene encoding the antigen-binding fragment and functional regions from other genes (e.g. enzymes). In the case of gene fusions, the two components are present within the same gene. Alternatively, antigen-binding fragments can be chemically bonded to the moiety by any of a variety of well known chemical procedures. For example, when the moiety is a protein, the linkage can be by way of homo- or hetero-bifunctional cross linkers, e.g., SPDP, SMCC, carbodiimide glutaraldehyde, or the like. The moieties can be covalently linked, or conjugated, through a secondary reagent, including, but not limited to, a second antibody, protein A, or a biotin-avidin complex. Paramagnetic moieties and the conjugation thereof to antibodies are well-known in the art. See, e.g., Miltenyi et al. (1990) Cytometry 11:231-238.

Methods of antibody production and isolation are well known in the art. See, for example, Harlow and Lane (1988) Antibodies: A Laboratory Manual, Cold Spring Harbor Laboratory, New York. General antibody purification methods include, but are not limited to, salt precipitation (for example, with ammonium sulfate); ion exchange chromatography (for example, on a cationic or anionic exchange column run at neutral pH and eluted with step gradients of increasing ionic strength); gel filtration chromatography (including gel filtration HPLC); and chromatography on affinity resins such as protein A, protein G, hydroxyapatite, or anti-Ig. Antigen-binding fragments can also be purified on affinity columns comprising DCs or an antigenic portion thereof. Preferably fragments are purified using Protein-A-CL-Sepharose™ 4B chromatography followed by chromatography on a DEAE-Sepharose™ 4B ion exchange column.

The anti-CD141 antigen binding fragments can be hybrid antibodies, in which one pair of H and L chains is obtained from a first antibody, while the other pair of H and L chains is obtained from a different second antibody. For purposes of this invention, one pair of L and H chains is from anti-DC antibody. In one example, each L-H chain pair binds different epitopes of a DC-specific antigen. Such hybrids can also be formed using humanized H or L chains. The invention also encompasses other bispecific antibodies such as those containing two separate antibodies covalently linked through their constant regions.

Other antigen-binding fragments include antibodies in which the H or L chain has been modified to provide additional properties. For instance, a change in amino acid sequence can result in reduced immunogenicity of the resultant polypeptide. The changes range from changing one or more amino acid residues to the complete redesign of a region such as a C region domain. Typical changes include, but are not limited to, those related to complement fixation, interaction with membrane receptors, and other effector functions. A recombinant antibody can also be designed to aid the specific delivery of a substance (such as a cytokine) to a cell. Also encompassed by the invention are peptides in which various Ig domains have been placed in an order other than that which occurs in nature.

The size of the antigen-binding fragments can be only the minimum size required to provide a desired function. It can optionally comprise additional amino acid sequence, either native to the antigen-binding fragment, or from a heterologous source, as desired. Anti-DC antigen-binding fragments can contain only 5 consecutive amino acid residues from an antibody V region sequence. Polypeptides comprising 7 amino acid residues, more preferably about 10 amino acid residues, more preferably about 15 amino acid residues, more preferably about 25 amino acid residues, more preferably about 50 amino acid residues, more preferably about 75 amino acid residues from the antibody L or H chain V region are also included. Even more preferred are polypeptides, comprising the entire antibody L or H chain V region.

Substitutions can range from changing or modifying one or more amino acid residue to complete redesign of a region, such as the V region. Amino acid residue substitutions, if present, are preferably conservative substitutions that do not deleteriously affect folding or functional properties of the peptide. Groups of functionally related amino acid residues within which conservative substitutions can be made are glycine/alanine; valine/isoleucine/leucine; asparagine/glutamine; aspartic acid/glutamic acid; serine/threonine/methionine; lysine/arginine; and phenylalanine/tyrosine/tryptophan. Antigen-binding fragments can be glycosylated or unglycosylated, can be modified post-translationally (e.g., acetylation, and phosphorylation) or can be modified synthetically (e.g., the attachment of a labeling group).

Polypeptide derivatives comprising both an L chain and an H chain can be formed as separate L and H chains and then assembled, or assembled in situ by an expression system for both chains. Such expression systems can be created by transfecting with a plasmid comprising separate transcribable regions for the L and H chain, or by co-transfecting the same cell with plasmids for each chain. In a third method, a suitable plasmid with an H chain encoding region is transfected into an H chain loss mutant.

H chain loss mutants can be obtained by treating anti-DC antibody producing cells with fluorescein-labeled rabbit anti-mouse IgG (H chain specific, DAKO Corporation, Carpinteria, Calif.) according to the supplier's instruction. The stained and unstained cell populations are analyzed by flow cytometry. Unstained cells are collected in a sterilized tube and placed in 96-well plates at 1 cell/well by limiting dilution. Culture supernatants are then assayed by ELISA using goat anti-mouse IgG (H chain specific) and goat anti-mouse kappa. Clones having a kappa-positive, IgG-negative phenotype are subcloned at least 3 times to obtain stable anti-DC^{(-H)} mutants. mRNA from putative H chain loss mutants can be isolated and the sequence of the L chain V region cDNA determined. Reverse PCR of the mRNA for the VH is performed with 2 sets of 5′- and 3′- primers, and used for cloning of anti-DC^{(-H)} cDNA. An H chain loss mutant yields no detectable DNA band with these primers. Transfection of the cells proceeds with a suitable H chain plasmid.

Another antigen-binding fragment derivative is an antibody in which the constant region of the H or L chain has been modified to provide additional properties. For instance, a change in amino acid sequence can result in altered immunogenicity of the resultant polypeptide. The changes range from one or more amino acid residues to the complete redesign of constant region domain. Changes contemplated affect complement fixation, interaction with membrane receptors, and other effector functions. A recombinant antibody can also be designed to aid the specific delivery of a substance (such as a lymphokine or an antigen or an antigenic peptide derived from a tumor, virus, parasite or bacteria, or tolerogen (autoantigen)) to a cell. Also encompassed by the invention are proteins in which various Ig domains have been placed in an order other than that which occurs in nature.

Single chain V region fragments (“scFv”) of anti-CD141 antibodies can also be used. Single chain V region fragments are made by linking L and/or H chain V regions by using a short linking peptide. Bird et al. (1988) Science 242:423-426. Any peptide having sufficient flexibility and length can be used as a linker in a scFv. Usually the linker is selected to have little to no immunogenicity. An example of a linking peptide is (GGGGS)₃, which bridges approximately 3.5 nm between the carboxy terminus of one V region and the amino terminus of another V region. Other linker sequences can also be used, and can provide additional functions, such as a for attaching to a drug or solid support or specific delivery of a substance (such as a lymphokine or an antigen or an antigenic peptide derived from a tumor, virus, parasite or bacteria, or tolerogen (autoantigen)) to a cell.

All or any portion of the H or L chain can be used in any combination. Typically, the entire V regions are included in the scFv. For instance, the L chain V region can be linked to the H chain V region. Alternatively, a portion of the L chain V region can be linked to the H chain V region, or portion thereof. Also contemplated are scFvs in which the H chain V region is from an antibody described herein, and the L chain V region is from another Ig. A biphasic, scFv can be made in which one component is an antigen-binding fragment and another component is a different polypeptide, such as a T cell epitope.

The scFvs can be assembled in any order, for example, V_{H}—linker—V_{L} or V_{L}—linker—V_{H}. There can be a difference in the level of expression of these two configurations in particular expression systems, in which case one of these forms can be preferred. Tandem scFvs can also be made, such as (X)—(linker)—(X)—(linker)—(X), in which X are scFvs, or combinations thereof with other polypeptides. In another embodiment, single chain antibody polypeptides have no linker polypeptide, or just a short, inflexible linker. Possible configurations are V_{L}—V_{H} and V_{H}—V_{L}. The linkage is too short to permit interaction between V_{L} and V_{H} within the chain, and the chains form homodimers with a V_{L}/V_{H} antigen-binding site at each end. Such molecules are referred to as “diabodies.”

ScFvs can be produced recombinantly or synthetically. For synthetic production of scFv, an automated synthesizer can be used. For recombinant production of scFv, a suitable plasmid-containing polynucleotide that encodes the scFv can be introduced into a suitable host cell, either eukaryotic, such as yeast, plant, insect or mammalian cells, or prokaryotic, such as Escherichia coli, and the expressed protein can be isolated using standard protein purification techniques. ScFvs can also be obtained from a phage display library.

A particularly useful system for the production of scFvs is plasmid pET-22b(+)(Novagen, Madison, Wis.). E. coli pET-22b(+) contains a nickel ion binding domain consisting of 6 sequential histidine residues, which allows the expressed protein to be purified on a suitable affinity resin. Another example of a suitable vector is pcDNA3 (Invitrogen, San Diego, Calif.).

Conditions of gene expression preferably ensure that the scFv assumes optimal tertiary structure. Depending on the plasmid used (especially promoter activity), and the host cell, it can be necessary to modulate production rate. For instance, use of a weaker promoter, or expression at lower temperatures, can be necessary to optimize production of properly folded scFv in prokaryotic systems; or, it can be used to express scFv in eukaryotic cells.

Polymeric forms of antigen-binding fragments, containing a plurality of DC-specific antigen-binding fragments can be used. One embodiment is a linear polymer of antigen-binding fragments, optionally conjugated to carrier. These linear polymers can comprise multiple copies of a single antigen-binding fragment polypeptide, or combinations of different polypeptides, and can have tandem polypeptides, or polypeptides separated by other amino acid sequences.

Another embodiment is multiple antigen peptides (MAPs). MAPs have a small immunologically inert core having radially branching lysine dendrites, onto which a number of antigen-binding fragment polypeptides are covalently attached. See for instance, Posnett et al. (1988) J. Biol. Chem. 263:1719-1725; and Tam (1989) Met. Enz. 168:7-15. The result is a large macromolecule having a high molar ratio of antigen-binding fragment polypeptides to core. MAPs are efficient immunogens and useful antigens for immunoassays. The core for creating MAPs can be made by standard peptide synthesis techniques, or obtained commercially (Quality Controlled Biochemicals, Inc., Hopkinton, Mass.). A typical core matrix is made up of three levels of lysine and eight amino acid residues.

Cancer patients are often immunosuppressed and tolerant to some tumor-associated antigens (TAA). Triggering an active immune response to such TAA represents an important challenge in cancer therapy. Immunization with a given antigen generates an immune response including a CTL response, preferably a strong CTL response. The production of antibodies against the antigen can be helpful if the tumor cells are killed by ADCC (antibody-dependent cellular cytotoxicity). The invention encompasses the use of DCs identified and isolated by use of the antigen-binding fragments of the invention in inducing specific immune responses by methods known in the art. The immune responses can be specific to any antigen including, without limitation, those associated with cancer, infectious viruses, infectious bacteria, infectious parasites, infectious yeast, and autoimmune diseases (the induce tolerance). The ability to isolate subpopulations that are uniquely suited to inducing such a response results in preparations of DCs that are more effective than mixtures of subpopulations. Hybrid cells (e.g. DC/tumor cell) could also be used as cancer-specific therapy. Modified cells (including, without limitation, activated, in vitro matured, modulated with respect to their T helper cell polarizing capacity (Th1 v Th2 v Th3/Th-R), and modulated with respect to their T cell stimulating or anergizing or deleting capacity) are likewise encompassed by the invention and include, but are not limited to, genetically modified or transfected cells and cells that have been incubated with peptides or proteins suitable for antigen presentation or for internalization. Subpopulations include, without limitation, a particular differentiation stage within one lineage and a separate lineage of differentiation.

The invention provides methods of isolating DCs, subpopulations thereof and mixtures thereof. The cells are selected using antigen-binding fragments specific for CD141 by any separation method known in the art. These include pharmaceutical and therapeutic compositions and any other composition containing the isolated cells. That is, cells isolated by a BDCA-specific antigen-binding fragments may be more than about 5%, at least about 10%, at least about 30%, at least about 50%, at least about 70%, at least about 80% BDCA⁺, or even more than about 90% BDCA⁺ or more than about 95% BDCA⁺. in an embodiment, the DC subpopulations isolated by the methods described herein are preferably substantially homogeneous. Of course, subsequent combinations of the cells with other DCs, or other hematopoietic cells can decrease the percentage of BDCA⁺ cells, such combinations are also encompassed by the invention.

Likewise, the DCs obtained by the methods described herein are suitable for use in any method of treatment known in the art include references here. DCs altered to achieve these methods are also encompassed by the invention. These methods include, but are not limited to:

a) therapy with isolated DCs to induce specific T cell tolerance (killing or anergy instead of stimulation) in autoimmune diseases, allergies, graft versus host disease (GvHD), allograft rejection. For instance, DCs specific for such T cells can be modified to contain lysis, inactivating or death-inducing moieties so as to specifically target the T cells involved in the unwanted immune response for instance by antigen labeling or genetic modification such as by CD95L transfection. DC specificity for T cells is primarily caused by presentation of the appropriate T cell epitopes (peptides) via MHC I and II. The particular subsets of DCs with tolerance-inducing functions can be administered directly to the patient. Peripheral tolerance can be mediated by DCs modified to induce deletion (killing), anergy and suppression/regulation of T cells;

b) immunomodulation therapy with isolated DCs to induce particular cytokine expression profiles in specific T cells. This is particularly useful to influence production of Th1 (cytokines for specific inflammatory immune responses), Th2 (cytokines for specific humoral immune responses) or Th3 (cytokines for specific immunosuppression) cytokines. In the case of allergies and asthma for instance, induction of a Th1 response may reduce or eliminate the symptom-producing Th2 response;

c) therapy with DCs presenting antigens including, but not limited to, tumor antigens, viral antigens and cellular antigens;

d) therapy with DCs (with or without presenting antigens) and various cofactors including, but not limited to cytokines, costimulatory molecules and effector molecules in amounts and under conditions sufficient to modulate the immune response; and

e) stimulating T cells in vitro to obtain antigen-specific T cells.

The antigen-binding fragments described herein are also suitable for a number of methods of treatment. These include, but are not limited to:

a) antibodies mimicking the ligand- or ligand-mediated immunotherapy for instance of DCs involved in autoimmunity or in vivo targeting of antigens or nucleic acids (viruses, plasmid DNA, RNA etc.) to DCs for optimal and selective uptake/transfection; and

b) immunomonitoring: e.g. enumeration and characterization of BDCA-3⁺ DCs in various diseases and upon mobilization e.g. with a proliferation inducing ligand, e.g. flt3-Ligand or G-CSF.

Any carrier not harmful to the host can be used for the DCs. Suitable carriers are typically large, slowly metabolized macromolecules such as proteins; polysaccharides (such as latex functionalized Sepharose, agarose, cellulose, cellulose beads and the like); polymeric amino acid residues (such as polyglutamic acid, polylysine, and the like); amino acid copolymers; and inactive virus particles or attenuated bacteria, such as Salmonella.

2. Methods of Obtaining Additional DC-specific Antigen-binding Fragments

Methods of generating new DC-specific antigen-binding fragments, as detailed below, include, but are not limited to: 1) employing phage display techniques by which cDNA encoding antibody repertoires are preferably amplified from lymphocyte or spleen RNA using PCR and oligonucleotide primers specific for species-specific V regions; 2) immunizing mammals with the antigen and generating polyclonal or mAbs; and 3) employing phage display to make antibodies without prior immunization by displaying on phage, very large and diverse V gene repertoires. See, generally Hoogenboom et al. (1998) Immunotechnol. 4:1-20. Preferably, for therapeutic purposes, if non-human antigen binding fragments are to be used, these can be humanized by any method known in the art.

The method described by Medez et al. (1997) Nature Genetics 18:410 can be used. Briefly, purified antigen, is used to immunize transgenic mice lacking the native murine antibody repertoire and instead having most of the human antibody V-genes in the germ line configuration. Human antibodies are subsequently produced by the murine B cells. The antibody genes are recovered from the B cells by PCR library selection or classic hybridoma technology.

Alternatively, antibodies can be obtained from mice (such as BALB/c) after injection with purified DC-specific antigen. mAbs are generated using standard hybridoma technology. Maiti et al. (1997) Biotechnol. Int. 1:85-93 (human hybridomas); and Kohler and Milstein (1975) Nature 256:495-497 (mouse hybridomas). Murine antibodies can be subsequently humanized for instance by the methods described by Rosok et al. (1996) J. Biol. Chem. 271:22611-22618; Baca et al. (1997) J. Biol. Chem. 272:10678-10684; Rader et al. Proc. Natl. Acad. Sci. USA 95:8910-8915; and Winter and Milstein (1991) Nature 349:293-299.

A phage display approach can also be used to rapidly generate human antibodies against DCs. This approach can employ the method described by Henderikx et al. (1998) Cancer Res. 58:4324-32. Antibody fragments displayed on phage are selected from a large naïve phage antibody/fragment library containing different single chain antibodies by separating those that bind to immobilized antigen or DCs. Human antibody fragments are selected from naive repertoires constructed either from germline V-domains or synthesized with many mutations (mutations are targeted either by homologous gene re-assortments or error prone PCR) in both the framework and CDR regions. Antigen-binding fragments specifically reactive with DCs can be identified by screening against tumor and normal cells as described herein in order to identify DC-specific antigen-binding fragments.

Affinity chromatography in which binding antibodies can be subtracted from non-binding antibodies has been established for some time. Nissim et al. (1994) EMBO J. 13:692-698; and Vaughan et al. (1996) Nat. Biotechnol. 14:309-314. Critical parameters affecting success are the number and affinity of antibody fragments generated against a particular antigen. Until recently, the production of large, diverse libraries remained somewhat difficult. Historically, scFv repertoires have been assembled directly from VH and VL RT-PCR products. RNA availability and the efficiency of RT-PCR were limiting factors of the number of V genes available. Also, assembly required ligating three fragments, namely VH and VL and the linker regions. Marks et al. (1991) J. Mol. Biol. 222:581-597.

An improved library construction method uses cloned VH and VL gene repertoires in separate plasmid vectors to provide a stable and limitless supply of material for scFv assembly. Sheets et al. (1998) Proc. Natl. Acad. Sci. USA 95:6175-6162. Also, the efficiency is increased by having DNA encoding the linker region at the 5′ end of the VL library. Therefore there are only two fragments to be ligated instead of three.

Anti-CD141-antigen-binding fragments can also be derived or manipulated. For example, the immunogenic activity of the V regions of the L and H chains can be screened by preparing a series of short polypeptides that together span the entire V region amino acid sequence. Using a series of polypeptides of 20 or 50 amino acid residues in length, each V region can be surveyed for useful functional properties. It is also possible to carry out a computer analysis of a protein sequence to identify potentially immunogenic polypeptides. Such peptides can then be synthesized and tested.

The antigen-binding fragments can be made by any suitable procedure, including proteolysis, recombinant methods or chemical syntheses. These methods are known in the art and need not be described in detail. Examples of proteolytic enzymes include, but are not limited to, trypsin, chymotrypsin, pepsin, papain, V8 protease, subtilisin, plasmin, and thrombin. Intact antigen-binding fragments can be incubated with one or more proteases simultaneously or sequentially. Alternatively, or in addition, intact antibody can be treated with disulfide reducing agents. Peptides can then be separated from each other by techniques known in the art, including but not limited to, gel filtration chromatography, gel electrophoresis, and reverse-phase HPLC.

Anti-CD141 antigen-binding fragments can also be made by expression from a polynucleotide encoding the peptide, in a suitable expression system by any method known in the art. Typically, polynucleotides encoding a suitable polypeptide are ligated into an expression vector under control of a suitable promoter and used to genetically alter the intended host cell. Both eukaryotic and prokaryotic host systems can be used. The polypeptide is then isolated from lysed cells or from the culture medium and purified to the extent needed for its intended use. Examples of prokaryotic host cells appropriate for use with this invention include E. coli, Bacillus subtilis and any other suitable host cell. Examples of eukaryotic host cells include, but are not limited to yeast, avian, insect, plant, and animal cells such as COS7, HeLa, and CHO cells.

Optionally, matrix-coated channels or beads and cell co-cultures can be included to enhance growth of antigen-binding fragment producing cells. For the production of large amounts of mAbs, it is generally more convenient to obtain ascitic fluid. The method of raising ascites generally comprises injecting hybridoma cells into an immunologically naive, histocompatible or immunotolerant mammal, especially a mouse. The mammal can be primed for ascites production by prior administration of a suitable composition; e.g., Pristane. The ascitic fluid is removed from the animal and processed to isolate antibodies.

Alternatively, antigen-binding fragments can be chemically synthesized using amino acid sequence data and other information provided in this disclosure, in conjunction with standard methods of protein synthesis. A suitable method is the solid phase Merrifield technique. Automated peptide synthesizers are commercially available, such as those manufactured by Applied Biosystems, Inc. (Foster City, Calif.).

Another method of obtaining anti-CD141 antigen-binding fragments is to immunize suitable host animals with CD141 and follow standard methods for polyclonal or mAb production and isolation. mAbs thus produced can be “humanized” by methods known in the art. The invention thus encompasses humanized mAbs.

In “humanized” antibodies at least part of the sequence has been altered from its initial form to render it more like human Igs. In one version, the H chain and L chain C regions are replaced with human sequence. This is a fusion polypeptide comprising an anti-DC V region and a heterologous Ig (C) region. In another version, the CDR regions comprise anti-DC amino acid sequences, while the V framework regions have also been converted human sequences. See, for example, EP 0329400. In a third version, V regions are humanized by designing consensus sequences of human and mouse V regions, and converting residues outside the CDRs that are different between the consensus sequences.

In making humanized antibodies, the choice of framework residues can aid in retaining high binding affinity. In principle, a framework sequence from any human antibody can serve as the template for CDR grafting; however, it has been demonstrated that straight CDR replacement into such a framework can lead to significant loss of antigen binding affinity. Glaser et al. (1992) J. Immunol. 149:2606; Tempest et al. (1991) Biotechnol. 9:266; and Shalaby et al. (1992) J. Exp. Med. 175:217-225. The more homologous a human antibody is to the original murine antibody, the less likely that the human framework will introduce distortions into the murine CDRs that could reduce affinity. Based on a sequence homology search against an antibody sequence database, the human antibody IC4 provides good framework homology to muM4TS.22, although other highly homologous human antibodies are suitable as well, especially κ L chains from human subgroup I or H chains from human subgroup III. Kabat et al. (1987). Various computer programs such as ENCAD predict the ideal sequence for the V region. Levitt et al. (1983) J. Mol. Biol. 168:595. The invention thus encompasses human antibodies with different V regions. It is within the skill of one in the art to determine suitable V region sequences and to optimize these sequences. Methods for obtaining antibodies with reduced immunogenicity are also described in U.S. Pat. No. 5,270,202 and EP 699,755.

In certain applications, such as when an antigen-binding fragment is expressed in a suitable storage medium such as a plant seed, the antigen-binding fragment can be stored without purification. Fiedler et al. (1995) Biotechnol. 13:1090-1093. For most applications, it is generally preferable that the polypeptide is at least partially purified from other cellular constituents. Preferably, the peptide is at least about 50% pure as a weight percent of total protein. More preferably, the peptide is at least about 50-75% pure. For clinical use, the peptide is preferably at least about 80% pure.

If the peptides are to be administered to an individual, preferably it is at least 80% pure, more preferably at least 90% pure, even more preferably at least 95% pure and free of pyrogens and other contaminants. In this context, the percent purity is calculated as a weight percent of the total protein content of the preparation, and does not include constituents which are deliberately added to the composition purification.

Anti-CD141 is also useful for detecting, enumerating and/or identifying DCs and subsets thereof, in a biological sample. The methods include obtaining a biological sample, contacting the sample with an antigen-binding fragment described herein under conditions that allow antibody-antigen-binding and detecting binding, if any, of the antibody to the sample as compared to a control, biological sample.

After a biological sample is suitably prepared, for instance by enriching for DC concentration or antigen concentration, it is mixed with excess antigen-binding fragments under conditions that permit formation of a complex between DCs or antigen and the antibody. The amount of complex formed or the number of complex bearing DCs then determined, and eventually compared with complexes formed with standard samples containing known amounts of target antigen in the range expected or known DC concentrations. Complex formation can be observed by immunoprecipitation or nephelometry, but it is generally more sensitive to employ a reagent labeled with such labels as radioisotopes like ¹²⁵I, enzymes like peroxidase and β-galactosidase, or fluorochromes like fluorescein. Methods of detecting cells and antigens are well known in the art. For cell detection, flow cytometry is particularly useful, with antigen, ELISA is preferred.

The specific recognition of an anti-DC antigen-binding fragment to an antigen can be tested by any immunoassay known in the art. Any form of direct binding assay is suitable. In one such assay, one of the binding partners, the antigen or the putative antigen-binding fragment, is labeled. Suitable labels include, but are not limited to, radioisotopes such as ¹²⁵I, enzymes such as peroxidase, fluorescent labels such as fluorescein, and chemiluminescent labels. Typically, the other binding partner is insolubilized (for example, by coating onto a solid phase such as a microtiter plate) to facilitate removal of unbound soluble binding partner. After combining the labeled binding partner with the unlabeled binding partner, the solid phase is washed and the amount of bound label is determined.

When used for immunotherapy, the antigen-binding fragments described herein can be unlabeled or labeled with a therapeutic agent as described herein and as known in the art. These agents can be coupled either directly or indirectly to the antigen-binding fragments of the invention. One example of indirect coupling is by use of a spacer moiety. These spacer moieties, in turn, can be either insoluble or soluble (Diener et al. (1986) Science 231:148) and can be selected to enable drug release at the target site. Examples of therapeutic agents that can be coupled to antigen-binding fragments for immunotherapy include, but are not limited to, antigens, including tumor antigens, viral antigens, bacterial antigens, parasite-derived antigens and autoantigens, bioresponse modifiers, drugs, radioisotopes, lectins, and toxins. Bioresponse modifiers include cytokines and chemokines which include, but are not limited to, IL-2, IL-3, IL-4, G-CSF, GM-CSF, IL-10, IL-12, TGF-β, MTP-AB, SDF-1, Lymphotactin, DC-CK1, Eotoxins, IP-10, TARC, Rantes, MIP-1α, MIP-1β, SLC, ITAC, MIE, MDC, MCP-1, TCA-3, MCP-2, -3, -4 and interferons. Interferons with which antigen-binding fragments can be labeled include IFN-α, IFN-β, and IFN-γ and their subtypes.

In using radioisotopically conjugated antigen-binding fragments for immunotherapy, certain isotypes can be more preferable than others depending on such factors as isotype stability and emission. If desired, cell population recognition by the antigen-binding fragment can be evaluated by the in vivo diagnostic techniques described below. In general, α and β particle-emitting radioisotopes are preferred in immunotherapy. For example, a high energy β emitter capable of penetrating several millimeters of tissue, such as ⁹⁰Y, can be preferable. On the other hand, a short range, high energy α emitter, such as ²¹²Bi, can be preferable. Examples of radioisotopes which can be bound to the antigen-binding fragments of the invention for therapeutic purposes include, but are not limited to, ¹²⁵I, ¹³¹I, ⁹⁰Y, ⁶⁷Cu, ²¹²Bi, ²¹¹At, ²¹²Pb, ⁴⁷Sc, ¹⁰⁹Pd, and ¹⁸⁸Re.

Lectins are proteins, usually isolated from plant material, which bind to specific sugar moieties. Many lectins are also able to agglutinate cells and stimulate lymphocytes. However, ricin is a toxic lectin that has been used immunotherapeutically. This is preferably accomplished by binding the α peptide chain of ricin, which is responsible for toxicity, to the antibody molecule to enable site specific delivery of the toxic effect.

Toxins are poisonous substances produced by plants, animals, or microorganisms that, in sufficient dose, are often lethal. Diphtheria toxin is a substance produced by Corynebacterium diphtheria which can be used therapeutically. This toxin consists of an α and β subunit which under proper conditions can be separated. The toxic A chain component can be bound to an antigen-binding fragment described herein and used for site specific delivery to a specific subset of DCs.

Recombinant methods are well known in the art. The practice of the invention employs, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (Gait, ed., 1984); “Animal Cell Culture” (Freshney, ed., 1987); “Methods in Enzymology” (Academic Press, Inc.); “Handbook of Experimental Immunology” (Wei & Blackwell, eds.); “Gene Transfer Vectors for Mammalian Cells” (Miller & Calos, eds., 1987); “Current Protocols in Molecular Biology” (Ausubel et al., eds., 1987); “PCR: The Polymerase Chain Reaction”, (Mullis et al., eds., 1994); and “Current Protocols in Immunology” (Coligan et al., eds., 1991). These techniques are applicable to the production of the polynucleotides and polypeptides, and, as such, can be considered in making and practicing the invention. Particularly useful techniques are discussed in the sections that follow.

Functionally equivalent variants and derivatives of CD141 and functionally equivalent fragments thereof that can enhance, decrease or not significantly affect properties of the polypeptides encoded thereby. These functionally equivalent variants, derivatives, and fragments may display the ability to specifically bind to their respective antibodies. For instance, changes that will not significantly affect properties of the encoded polypeptide include, but are not limited to changes in a DNA sequence that do not change the encoded amino acid sequence, as well as those that result in conservative substitutions of amino acid residues, one or a few amino acid residue deletions or additions, and substitution of amino acid residues by amino acid analogs. Conservative substitutions are glycine/alanine; valine/isoleucine/leucine; asparagine/glutamine; aspartic acid/glutamic acid; serine/threonine/methionine; lysine/arginine; and phenylalanine/tyrosine/tryptophan.

The polynucleotides can comprise additional sequences, such as additional encoding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as can be desirable.

A polynucleotide can be of at least about 15 consecutive nucleotides, preferably at least about 20 nucleotides, more preferably at least about 25 consecutive nucleotides, more preferably at least about 35 consecutive nucleotides, more preferably at least about 50 consecutive nucleotides, even more preferably at least about 75 nucleotides, still more preferably at least about 100 nucleotides, still more preferably at least about 200 nucleotides, and even more preferably at least about 300 nucleotides that forms a stable hybrid with a polynucleotide encoding CD141. Any set of conditions can be used for this test, provided at least one set exists where the test polynucleotide demonstrates the required specificity.

Hybridization reactions can be performed under conditions of different “stringency”. Conditions that increase stringency of a hybridization reaction are published. See, for example, Sambrook and Maniatis. Examples of relevant conditions include (in order of increasing stringency): incubation temperatures of 25° C., 37° C., 50° C. and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water.

Polynucleotides can be covalently linked with a detectable label. Such polynucleotides are useful, for example, as probes for detection of related nucleotide sequences.

The polynucleotides can be obtained using chemical synthesis, recombinant cloning methods, PCR, or any combination thereof. Methods of chemical polynucleotide synthesis are well known in the art and need not be described in detail herein. One of skill in the art can use the sequence data provided herein to obtain a desired polynucleotide by employing a DNA synthesizer or ordering from a commercial service.

Alternatively, nucleotides encoding CD141 and the peptides encoded thereby can be obtained from a producing cell line, cloning vector, or expression vector. RNA or DNA encoding the desired sequence can be isolated, amplified, and processed by standard recombinant techniques. Such techniques include digestion with restriction nucleases, and amplification by polymerase chain reaction (PCR), or a suitable combination thereof. PCR technology is described in U.S. Pat. Nos. 4,683,195; 4,683,202; 4,754,065; and 4,800,159 as well as PCR: The Polymerase Chain Reaction, Mullis et al. eds., Birkauswer Press, Boston (1994). Isolation and purification of the peptides encoded thereby can be by any method known in the art.

Polynucleotides comprising a desired sequence can be inserted into a suitable vector, the vector in turn can be introduced into a suitable host cell for replication and amplification. Polynucleotides can be inserted into host cells by any means known in the art. Cells are transformed by introducing an exogenous polynucleotide by direct uptake, endocytosis, transfection, f-mating or electroporation. Once introduced, the exogenous polynucleotide can be maintained within the cell as a non-integrated vector (such as a plasmid) or integrated into the host cell genome. Amplified DNA can be isolated from the host cell by standard methods. See, e.g., Sambrook et al. (1989). RNA can also be obtained from transformed host cell, it can be obtained by using a DNA-dependent RNA polymerase.

Vectors encoding CD141 can be used for expression of recombinant polypeptides as well as a source of polynucleotides. Cloning vectors can be used to obtain replicate copies of the polynucleotides, or for storing the polynucleotides in a depository for future recovery. Expression vectors (and host cells containing these expression vectors) can be used to obtain polypeptides produced from the polynucleotides they contain. They can also be used where it is desirable to express CD141 in an individual and thus have intact cells capable of synthesizing the polypeptide, such as in gene therapy. Suitable cloning and expression vectors include any known in the art e.g., those for use in bacterial, mammalian, yeast and insect expression systems. Specific vectors and suitable host cells are known in the art and are not described in detail herein. See e.g. Gacesa and Ramji, Vectors, John Wiley & Sons (1994).

Cloning and expression vectors typically contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will grow under selective conditions. Typical selection genes either: (a) confer resistance to antibiotics or other toxic substances, e.g., ampicillin, neomycin, methotrexate; (b) complement auxotrophic deficiencies; or (c) supply critical nutrients not available from complex media. The choice of the proper marker gene will depend on the host cell, and appropriate genes for different hosts are known in the art. Vectors also typically contain a replication system recognized by the host.

Suitable cloning vectors can be constructed according to standard techniques, or can be selected from a large number of cloning vectors available in the art. While the cloning vector selected can vary according to the host cell intended to be used, useful cloning vectors will generally have the ability to self-replicate, can possess a single target for a particular restriction endonuclease, or can carry genes for a marker that can be used in selecting clones containing the vector. Suitable examples include plasmids and bacterial viruses, e.g., pUC18, mp18, mp19, pBR322, pMB9, ColE1, pCR1, RP4, phage DNAs, and shuttle vectors such as pSA3 and pAT28. These and many other cloning vectors are available from commercial vendors such as BioRad, Stratagene, and Invitrogen.

Expression vectors generally are replicable polynucleotide constructs that contain a polynucleotide encoding a BDCA of interest. The polynucleotide encoding BDCA is operatively linked to suitable transcriptional controlling elements, such as promoters, enhancers and terminators. For expression (i.e., translation), one or more translational controlling elements are also usually required, such as ribosome binding sites, translation initiation sites, and stop codons. These controlling elements (transcriptional and translational) can be derived from a gene encoding a BDCA, or they can be heterologous (i.e., derived from other genes or other organisms). A polynucleotide sequence encoding a signal peptide can also be included to allow a BDCA to cross or lodge in cell membranes or be secreted from the cell. A number of expression vectors suitable for expression in eukaryotic cells including yeast, avian, and mammalian cells are known in the art. One example of an expression vector is pcDNA3 (Invitrogen, San Diego, Calif.), in which transcription is driven by the cytomegalovirus (CMV) early promoter/enhancer. This vector also contains recognition sites for multiple restriction enzymes for insertion of the polynucleotide of interest. Another example of an expression vector (system) is the baculovirus/insect system. Other suitable for use in antibody-targeted gene therapy comprising a polynucleotide encoding a BDCA. Suitable systems are described for instance by Brown et al. (1994) Virol. 198:477-488; and Miyamura et al. (1994) Proc. Natl. Acad. Sci. USA 91:8507-8511.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection. The choice of means of introducing vectors or polynucleotides encoding BDCAs will often depend on features of the on the host cell.

Once introduced into a suitable host cell, expression of a BDCA can be determined using any assay known in the art. For example, the presence thereof can be detected by RIA or ELISA of the culture supernatant (if the polypeptide is secreted) or cell lysates.

A vector of this invention can contain one or more polynucleotides encoding a BDCA. It can also contain polynucleotide sequences encoding other polypeptides that enhance, facilitate, or modulate the desired result, such as cytokines, including, but not limited to, IL-2, IL-4, GM-CSF, TNF-α and IFN-γ. Also embodied in this invention are vaccinia vectors encoding for recombinant BDCAs.

Other embodiments of this invention are host cells transformed with polynucleotides encoding BDCAs and vectors comprising the polynucleotide sequences, as described above. Both prokaryotic and eukaryotic host cells can be used. Prokaryotic hosts include, but are not limited to, bacterial cells, for example E. coli and mycobacteria. Eukaryotic hosts include, but are not limited to, yeast, insect, avian, plant and mammalian cells. Host systems are known in the art and need not be described in detail herein. Examples of a mammalian host cells include, but are not limited to, CHO and NS0, obtainable from the European Collection of Cell Cultures (England). Transfection of NS0 cells with a plasmid, for example, which is driven by a CMV promoter, followed by amplification of this plasmid in using glutamine synthetase provides a useful system for protein production. Cockett et al. (1990) Bio/Technology 8:662-667.

The polynucleotides have several uses. They are useful, for example, in expression systems for the production of CD141. Further, the polynucleotides are also useful as primers to effect amplification of desired polynucleotides. The polynucleotides are also useful in pharmaceutical compositions including vaccines and for gene therapy.

The polynucleotides can also be used as hybridization probes. Suitable hybridization samples include cells transformed ex vivo for use in gene therapy. In one illustration, DNA or RNA is extracted from a sample, and optionally run on a gel and/or digested with restriction nucleases. The processed sample polynucleotide is typically transferred to a medium suitable for washing. The sample polynucleotide is then contacted with the CD141-encoding polynucleotide probe under conditions that permit a stable duplex to form if the sample contains a complementary polynucleotide sequence. Any stable duplexes formed are detected by any suitable means. For example, the polynucleotide probe can be supplied in labeled form, and label remaining with the sample after washing will directly reflect the amount of stable duplex formed. In a second illustration, hybridization is performed in situ. A suitably prepared tissue sample is overlaid with a labeled probe to indicate the location of BDCA-encoding sequences.

A short polynucleotide can also be used as a primer for a PCR reaction, particularly to amplify a longer sequence comprising a region hybridizing with the primer. This can be conducted preparatively, in order to produce polynucleotide for further genetic manipulation. It can also be conducted analytically, to determine whether a BDCA-encoding polynucleotide is present, for example, in a sample of diagnostic interest.

Another use of the polynucleotides is in vaccines and gene therapy. The general principle is to administer the polynucleotide so that it either promotes or attenuates the expression of the polypeptide encoded thereby. Thus, methods of inducing an immune response and methods of treatment comprise administration of an effective amount of polynucleotides encoding CD141 to an individual. In these methods, a polynucleotide encoding BDCA is administered to an individual, either directly or via cells transfected with the polynucleotide. Preferably, the polynucleotide is in the form of a circular plasmid, preferably in a supercoiled configuration. Preferably, the polynucleotide is replicated inside a cell. Thus, the polynucleotide is operatively linked to a suitable promoter, such as a heterologous promoter that is intrinsically active in cells of the target tissue type. Preferably, once in cell nuclei, plasmids persist as circular non-replicating episomal molecules. In vitro mutation can be carried out with plasmid constructs to encode, for example, molecules with greater affinity and/or avidity.

To determine whether plasmids containing CD141 polynucleotides are capable of expression in eukaryotic cells, cells such as COS-7, CHO, or HeLa can be transfected with the plasmids. Expression is then determined by immunoassay; for example, by Western blot. Further characterization of the expressed polypeptide can be achieved by purifying the peptide and then conducting one of the functional assays described herein.

3. Enumeration of BDCA-3 Cells

In one aspect, as described elsewhere herein, the invention provides a method of distinguishing a subpopulation of dendritic cells from at least one other cell in a population of peripheral blood cells by contacting the population with an CD141 binding fragment and detecting the cells to which the binding fragment binds. In some embodiments, the CD141 binding fragment is detectably labeled (e.g., with a fluorescent label). One convenient method for enumeration is by flow cytometry (see Examples). In one embodiment, the method involves contacting said population with at least one antibody (other than anti-CD141) with specificity for a dendritic cell lineage marker or at least one antibody with specificity for a non-dendritic cell lineage marker and detecting the cells to which the non-CD141 antibody binds.

In another example, the cells may be quantitated by in situ hybrdization with a labeled polynucleotide that hybridizes to CD141 mRNA (see GenBank). In situ hybridization assays are well known and are generally described in Angerer et al., Methods Enzymol., 152: 649-660 (1987) and Ausubel, supra. The method usually involves initially fixing test cells to a support (e.g., the walls of a microtiter well) and then permeabilizing the cells with an appropriate permeabilizing, solution. A solution containing labeled probes for CD141 mRNA is then contacted with the cells and the probes allowed to hybridize with the nucleic acids. Excess probe is digested, washed away and the amount of hybridized probe measured. This approach is described in greater detail by Harris, 1996, Anal. Biochem. 243:249-256; Singer et al., 1986, Biotechniques 4:230-250; Haase et al., 1984, Methods In Virology, vol. VII, pp. 189-226; and Nucleic Acid Hybridization: A Practical Approach (Hames, et al., eds., 1987). In an other example, the cells in the enriched fraction may be quantitated by morphology.

4. Dendritic Cell Cytokine Production

In related aspects, the invention provides a method of monitoring production of dendritic cell cytokines by a dendritic cell by culturing a cell from an enriched dendritic cell composition, and monitoring cytokines produced by dendritic cell. Method for detecting and measuring cytokine production are known (e.g., ELISPOT). One exemplary method is described in copending U.S. patent application 08/441,259 (“Direct Selection Of Cells By Secretion Product”). Any secretion product of a dendritic cell can be assayed, such as, for example, IL-1, IL-12, IL-18, interferons alpha, beta, gamma. In another aspect, the invention provides a method of assaying for the effect of an agent on dendritic cell cytokine production comprising contacting an enriched dendritic cell composition (or cell from such a compositon) with the agent and monitoring the effect of the agent on cytokines produced by dendritic cells in the composition. Exemplary modulating agents include antibodies, ligands, cytokines, hormones, chemical agents, drugs and the like. Modulating agents can be obtained from libraries, such as natural product libraries or combinatorial libraries. In each case, the effect of an agent on dendritic cell function can be tested using the compositions of the invention. In one embodiment the invention provides a method of modulating cytokine production by a dendritic cell by contacting an enriched (e.g., substantially purified) composition of BDCA-3 dendritic cells with an agent that modulates dendritic cell cytokine production. In an embodiment, the agent induces expression of type I interferon, IL-12 or IL-4.

5. Therapeutic Compositions

A. Compositions of Matter

The preparation of pharmaceutical compositions described herein is conducted in accordance with generally accepted procedures for the preparation of pharmaceutical preparations. See, for example, Remington's Pharmaceutical Sciences 18th Edition (1990), E. W. Martin ed., Mack Publishing Co., PA. Depending on the intended use and mode of administration, it can be desirable to process the active ingredient further in the preparation of pharmaceutical compositions. Appropriate processing can include sterilizing, mixing with appropriate non-toxic and non-interfering components, dividing into dose units, and enclosing in a delivery device. In one embodiment, the therapeutic compositions contain DCs, subpopulations thereof or mixtures thereof. In another embodiment, the compositions contain the antigen-binding fragments described herein. Preferably, the antigen-binding fragments are, or are derived from, the mAbs listed in Table 1. Preferably the DC compositions contain DCs isolated with one of these antigen-binding fragments.

(a) General Modes of Administration

Pharmaceutical compositions are administered by a mode appropriate for the form of composition. Typical routes include intravenous, subcutaneous, intramuscular, intraperitoneal, intradermal, oral, intranasal, intradermal, and intrapulmonary (i.e., by aerosol). Pharmaceutical compositions for human use are typically administered by a parenteral route, most typically intravenous, subcutaneous, intramuscular. Although not required, pharmaceutical compositions are preferably supplied in unit dosage form suitable for administration of a precise amount. Also contemplated by this invention are slow release or sustained release forms, whereby a relatively consistent level of the active compound are provided over an extended period.

(b) Liquid Formulations

Liquid pharmaceutically acceptable compositions can, for example, be prepared by dissolving or dispersing a polypeptide or polynucleotide embodied herein in a liquid excipient, such as water, saline, aqueous dextrose, glycerol, or ethanol. The composition can optionally also contain other medicinal agents, pharmaceutical agents, carriers, and auxiliary substances such as wetting or emulsifying agents, and pH buffering agents. Compositions for injection can be supplied as liquid solutions or suspensions, as emulsions, or as solid forms suitable for dissolution or suspension in liquid prior to injection.

Pharmaceutical compositions for oral, intranasal, or topical administration can be supplied in solid, semi-solid or liquid forms, including tablets, capsules, powders, liquids, and suspensions. For administration via the respiratory tract, a preferred composition is one that provides a solid, powder, or liquid aerosol when used with an appropriate aerosolizer device.

Compositions can comprise liposomes with membrane bound peptide to specifically deliver the liposome to the area of the tumor or neoplastic cells or to the immune system. These liposomes can be produced such that they contain, in addition to peptide, immunotherapeutic agents such as those described above which would then be released at the recognition site. Wolff et al. (1984) Biochem. Biophys. Acta 802:259. Another such delivery system utilizes chimeric parvovirus B19 capsids for presentation of the antigen-binding fragments. Brown et al. (1994); and Miyamura et al. (1994). Such chimeric systems are encompassed for use herein.

Compositions can be assessed for their efficacy in a number of ways. Accordingly, test compounds are prepared as a suitable pharmaceutical composition and administered to test subjects. Initial studies are preferably done in small animals such as mice or rabbits, optionally next in non-human primates and then ultimately in humans. Immunogenicity is preferably tested in individuals without a previous antibody response. A test composition in an appropriate test dose is administered on an appropriate treatment schedule. It can be appropriate to compare different doses and schedules within the predicted range. The dosage ranges for the administration of antigen-binding fragments are large enough to produce the desired effect in which the symptoms of the disease are ameliorated without causing undue side effects such as unwanted cross-reactions and anaphylactic reactions. Generally, the dosage will vary with the age, condition, sex and extent of the disease in the patient and can be determined by one of skill in the art. The dosage can be adjusted by the individual physician in the event of any complication. Generally, when the compositions are administered conjugated with therapeutic agents, lower dosages, comparable to those used for in vivo immunodiagnostic imaging, can be used.

B. Antigen-binding Fragments

The antigen-binding fragments described herein are suitable for use in pharmaceutical compositions. Such pharmaceutical compositions are useful for inducing or aiding an immune response and treating neoplastic diseases, or including tolerance and treating autoimmune diseases, (GvHD, allograft rejection, allergen, etc.) either alone or in conjunction with other forms of therapy, such as chemotherapy, radiotherapy or immune therapies described in WO98/23735; WO98/34642; WO97/10000; WO97/10001; and WO97/06821. Other methods of treatment are described herein and/or known in the art. Suitable diseases include, without limitation, viral, parasitic, bacterial, fungal, neoplastic and autoimmune.

In a murine breast cancer model, Flt3-Ligand (Flt3-L), a stimulatory cytokine for a variety of hematopoietic lineages, including DCs and B cells, has been used in conjunction with murine breast cancer cells as a vaccine. Chen et al. (1997) Cancer Res. 57:3511-6. DCs can also be loaded with or transduced to express tumor antigens; these cells are then used as adjuvants to tumor vaccination. DCs present tumor-associated antigens endogenously to the afferent lymphatic system in the appropriate MHC-restricted context. Wan et al. (1997) Hum. Gene Ther. 8:1355-63; Peiper et al. (1997) Surgery 122:235-41; and Smith et al. (1997) Int. Immunol. 9:1085-93. Current melanoma vaccines manipulate antigen presentation networks and combine the best cellular and antibody anti-tumor immune response effective in mediating tumor protective immunity. These therapies have caused regression, delayed disease progression or an improvement in survival in some cases, with a paucity of side effects. Kuhn et al. (1997) Dermatol. Surg. 23:649-54. Melanoma vaccines are also reviewed in Conforti et al. (1997) J. Surg. Oncol. 66:55-64.

Vaccines can be packaged in pharmaceutically acceptable carriers, admixed with adjuvants or other components (such as cytokines) as known in the art. Vaccines for veterinarian use are substantially similar to that in humans with the exception that adjuvants containing bacteria and bacterial components such as Freund's complete or incomplete adjuvants, are allowed in the formulations.

6. Methods of Treatment

A variety of disorders as described herein and/or known in the art can be treated with the antigen binding compositions. The methods comprise administering an amount of a pharmaceutical composition containing a composition of the invention in an amount effective to achieve the desired effect, be it palliation of an existing condition or prevention of recurrence. For treatment of cancer, the amount of a pharmaceutical composition administered is an amount effective in producing the desired effect. An effective amount can be provided in one or a series of administrations. An effective amount can be provided in a bolus or by continuous perfusion. Suitable active agents include the anti-neoplastic drugs, bioresponse modifiers and effector cells such as those described by Douillard et al. (1986) Hybridomas (Supp.) 1:5139.

Pharmaceutical compositions and treatment modalities are suitable for treating a patient by either directly or indirectly eliciting an immune response against neoplasia. An “individual,” “patient” or “subject” is a vertebrate, preferably a mammal, more preferably a human. Mammals include, but are not limited to: humans, wild animals, feral animals, farm animals, sport animals, and pets. A “cancer subject” is a mammal, preferably a human, diagnosed as having a malignancy or neoplasia or at risk thereof.

As used herein, “treatment” refers to clinical intervention in an attempt to alter the disease course of the individual or cell being treated, and can be performed either for prophylaxis or during the course of clinical pathology. Therapeutic effects of treatment include without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis.

The “pathology” associated with a disease condition is any condition that compromises the well-being, normal physiology, or quality of life of the affected individual. This can involve, but is not limited to, destructive invasion of affected tissues into previously unaffected areas, growth at the expense of normal tissue function, irregular or suppressed biological activity, aggravation or suppression of an inflammatory or immunologic response, increased susceptibility to other pathogenic organisms or agents, and undesirable clinical symptoms such as pain, fever, nausea, fatigue, mood alterations, and such other disease-related features as can be determined by an attending physician.

An “effective amount” is an amount sufficient to effect a beneficial or desired clinical result upon treatment. An effective amount can be administered to a patient in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition and the form and effective concentration of the antigen-binding fragment administered.

The term “immunomodulatory” or “modulating an immune response” as used herein includes immunostimulatory as well as immunosuppressive effects. Immunostimulatory effects include, but are not limited to, those that directly or indirectly enhance cellular or humoral immune responses. Examples of immunostimulatory effects include, but are not limited to, increased antigen-specific antibody production; activation or proliferation of a lymphocyte population such as NK cells, CD4⁺ cells, CD8⁺ cells, macrophages and the like; increased synthesis of cytokines or chemokines including, but not limited to, IL-1, IL-2, IL-4, IL-5, IL-6, IL-12, interferons, TNF-α, IL-10, TGF-β and the like. Immunosuppressive effects include those that directly or indirectly decrease cellular or humoral immune responses. Examples of immunosuppressive effects include, but are not limited to, a reduction in antigen-specific antibody production such as reduced IgE production; activation of lymphocyte or other cell populations that have immunosuppressive activities such as those that result in immune tolerance; and increased synthesis of cytokines that have suppressive effects toward certain cellular functions including, but not limited to IL-10 and TGF-β. One example of this is IFN-γ, which appears to block IL-4 induced class switch to IgE and IgG1, thereby reducing the levels of these antibody subclasses.

Suitable human subjects for cancer therapy further comprise two treatment groups, which can be distinguished by clinical criteria. Patients with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in this invention is administered to these patients to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of cancer, but have been responsive to another mode of therapy. The prior therapy can have included (but is not restricted to, surgical resection, radiotherapy, and traditional chemotherapy. As a result, these individuals have no clinically measurable tumor. However, they are suspected of being at risk for progression of the disease, either near the original tumor site, or by metastases.

“Adjuvant” as used herein has several meanings, all of which will be clear depending on the context in which the term is used. In the context of a pharmaceutical preparation, an adjuvant is a chemical or biological agent given in combination (whether simultaneously or otherwise) with, or recombinantly fused to, an antigen to enhance immunogenicity of the antigen. For review see, Singh et al. (1999) Nature Biotech. 17:1075-1081. Isolated DCs have also been suggested for use as adjuvants. Compositions for use therein are included in this invention. In the context of cancer diagnosis or treatment, adjuvant refers to a class of cancer patients with no clinically detectable tumor mass, but who are suspected of risk of recurrence.

This group can be further subdivided into high-risk and low-risk individuals. The subdivision is made on the basis of features observed before or after the initial treatment. These features are known in the clinical arts, and are suitably defined for each different cancer. Features typical of high-risk subgroups are those in which the tumor has invaded neighboring tissues, or who show involvement of lymph nodes.

Another suitable group is those with a genetic predisposition to cancer but who have not yet evidenced clinical signs of cancer. For instance, women testing positive for a genetic mutation associated with breast cancer, but still of childbearing age, can wish to receive one or more of the antigen-binding fragments described herein in treatment prophylactically to prevent the occurrence of cancer until it is suitable to perform preventive surgery.

Human cancer patients, including, but not limited to, glioblastoma, melanoma, neuroblastoma, adenocarcinoma, glioma, soft tissue sarcoma, and various carcinomas (including small cell lung cancer) are especially appropriate subjects. Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bileduct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas.

The patients can have an advanced form of disease, in which case the treatment objective can include mitigation or reversal of disease progression, and/or amelioration of side effects. The patients can have a history of the condition, for which they have been treated, in which case the therapeutic objective will typically include a decrease or delay in the risk of recurrence.

Autoimmune disorders are the caused by a misdirected immune response resulting in self-destruction of a variety of cells, tissues and organs. The cause of these disorders is unknown. Recognition of self through the MHC is known to be of importance in an immune response. However, prevention of an autoimmune response and the cells responsible for autoimmunity are not well understood.

Autoimmunity results from a combination of factors, including genetic, hormonal, and environmental influences. Many autoimmune disorders are characterized by B cell hyperactivity, marked by proliferation of B cells and autoantibodies and by hypergammaglobulinemia. B cell hyperactivity is probably related to T cell abnormalities. Hormonal and genetic factors strongly influence the incidence of autoimmune disorders; for example, lupus erythematosus predominantly affects women of child-bearing age, and certain HLA haplotypes are associated with an increased risk of specific autoimmune disorders.

Common autoimmune disorders include, but are not limited to, rheumatoid arthritis, juvenile rheumatoid arthritis, psoriatic arthritis, ankylosing spondylitis, Sjögren's syndrome, lupus erythematosus, Goodpasture's syndrome, Reiter's syndrome, scleroderma, vasculitis, polymyositis and dermatomyositis. Many of these conditions include aberrant inflammatory reactions related to the immunologic disorders. The DCs described herein are suitable for use in treatment of these disorders particularly when used to inactivate or induce tolerogenization in T cells involved in the disorder. Methods of treatment are known in the art. As discussed herein, one or more of the subsets of DCs obtained by the methods described herein are suitable for use in treatment of autoimmunity.

“Immunologic activity” of an antigen-binding fragment refers to specifically binding the antigen which the intact antibody recognizes. Such binding can or can not elicit an immune response. A specific immune response can elicit antibody, B cell responses, T cell responses, any combination thereof, and effector functions resulting therefrom. Included, without limitation, are the antibody-mediated functions ADCC and complement-mediated cytolysis (CDC). The T cell response includes, without limitation, T helper cell function, cytotoxic T cell function, inflammation/inducer T cell function, and T cell mediated immune suppression. A compound (either alone or in combination with a carrier or adjuvant) able to elicit either directly or indirectly, a specific immune response according to any of these criteria is referred to as “immunogenic.” Antigen-binding fragment “activity” or “function” refers to any of the immunologic activities of an antibody, including detection, amelioration or palliation of cancer.

An “immune response” refers to induction or enhancement of an immunologic response to malignant or diseased tissue, disease-causing agents and other foreign agents to which the body is exposed. Immune responses can be humoral, as evidenced by antibody production; and/or cell-mediated, as evidenced by cytolytic responses demonstrated by such cells as natural killer cells or cytotoxic T lymphocytes (CTLs) and the cytokines produced thereby. Immune responses can be monitored by a mononuclear cell infiltrate at the site of infection or malignancy. Typically, such monitoring is by histopathology. A “cancer-specific immune response” is one that occurs against the malignancy but not against non-cancerous cells. The treatments described herein typically induce or augment a cell-mediated immune response but can also induce or augment an antibody-mediated immune response. The treatments can also influence the type of immune response to the antigen.

The compositions according to the invention are also suitable for use in inducing an antigen-specific Th1 immune response. Stimulating a Th1-type immune response can be measured in a host treated in accordance with the invention and can be determined by any method known in the art including, but not limited to, a reduction in levels of IL-4 measured before and after antigen challenge; or detection of lower (or even absent) levels of IL-4 in a treated host as compared to an antigen-primed, or primed and challenged, control treated without the compositions of the invention; an increase in levels of IL-12, IL-18 and/or IFN (α, β or γ, preferably IFN-γ in a treated host as compared to an antigen-primed or primed and challenged control; IgG2a antibody production in a treated host as compared to an untreated control; a reduction in levels of antigen-specific IgE as measured before and after antigen challenge or detection of lower (or even absent) levels of antigen-specific IgE in a treated host as compared to an antigen primed or primed and challenged untreated host. A variety of these determinations can be made by measuring cytokines made by APCs and/or lymphocytes, preferably DCs and/or T cells, in vitro or ex vivo using methods described herein and known in the art. Methods to determine antibody production include any known in the art.

The Th1 biased cytokine induction produces enhanced cellular immune responses, such as those performed by NK cells, cytotoxic killer cells, Th1 helper and memory cells. These responses are particularly beneficial for use in protective or therapeutic vaccination against viruses, fungi, protozoan parasites, bacteria, allergic diseases and asthma, as well as tumors.

The invention further encompasses screening for suitable moieties for interfering with ligation of BDCA-3 and compositions of these moieties.

When antigen-binding fragments are used in combination with various therapeutic agents, the administration of both usually occurs substantially contemporaneously. The term “substantially contemporaneously” means that they are administered reasonably close together with respect to time. The administration of the therapeutic agent can be daily, or at any other suitable interval, depending upon such factors, for example, as the nature of the ailment, the condition of the patient and half-life of the agent.

Therapeutic compositions can be administered by injection or by gradual perfusion over time. The antigen-binding fragments can be administered intravenously, intraperitoneally, intramuscularly, subcutaneously, intracavity, intranodal, intrathecally or transdermally, alone or in combination with other therapeutic agents.

Another method of administration is intralesionally, for instance by injection directly into the tumor. Intralesional administration of various forms of immunotherapy to cancer patients does not cause the toxicity seen with systemic administration of immunologic agents. Fletcher et al. (1987) Lymphokine Res. 6:45; Rabinowich et al. (1987) Cancer Res. 47:173-177; Rosenberg et al. (1989) Science 233:1318; and Pizz et al. (1984) J. Int. Cancer 34:359.

Further, it can be desirable to administer the compositions locally to the area in need of treatment; this can be achieved by, for example, local infusion during surgery, by injection, by means of a catheter, or by means of an implant, the implant being of a porous, non-porous, or gelatinous material, including membranes, such as silastic membranes, or fibers. A suitable such membrane is Gliadel® provided by Guilford sciences.

Regarding functional modulation of DC, the following aspects are encompassed by the invention:

a) Induction and down-regulation of CD4⁺ and CD8⁺ T cells responses.

b) Polarization of the immune response towards tolerance or immunity

c) Polarization of CD4⁺ T cell responses towards Th1 cell development, Th2 cells development or Th3/T-regulatory-1 CD4⁺ T cell development. The latter down-regulate immune responses, possibly via secretion of TGF-β and/or IL-10.

d) DC are usually thought of as antigen-presenting cells for T cells. However, recent studies from several laboratories have shown that they have important roles in B-cell activation and regulation of antibody synthesis. B cell responses can therefore be modulated via BDCA-2 on DCs. The same can also be true for NK cell responses.

In an aspect of the invention a method of inhibiting an interaction of a dendritic cell and a T cell by contacting the dendritic cell with a CD141 binding fragment or reducing the expression of CD141 on the dendritic cell surface is provided. In an aspect, the invention provides a method of treating inflammation by administering to a subject in need thereof an amount of an agent that inhibits the interaction of CD141 and a T cell or reduces the expression of CD141 on the surface of dendritic cells. The agent is administered in vivo or in vitro.

7. Kits

A kit may be provided for the practice of the methods disclosed herein. For example, in one embodiment, a kit for enriching dendritic cells from a mixture of cells is provided containing at least one anti-CD141 antibody (e.g., binding fragment) conjugated to a magnetic microparticle. Optionally the kit also contains a detectably labelled antibody that does not bind BDCA-3⁺ dendritic cells. The kit can also inlcude a column for magnetic separation. The kit may further include buffers and other reagents used in the separation process.

### EXAMPLE 1

### Cloning of BDCA-3

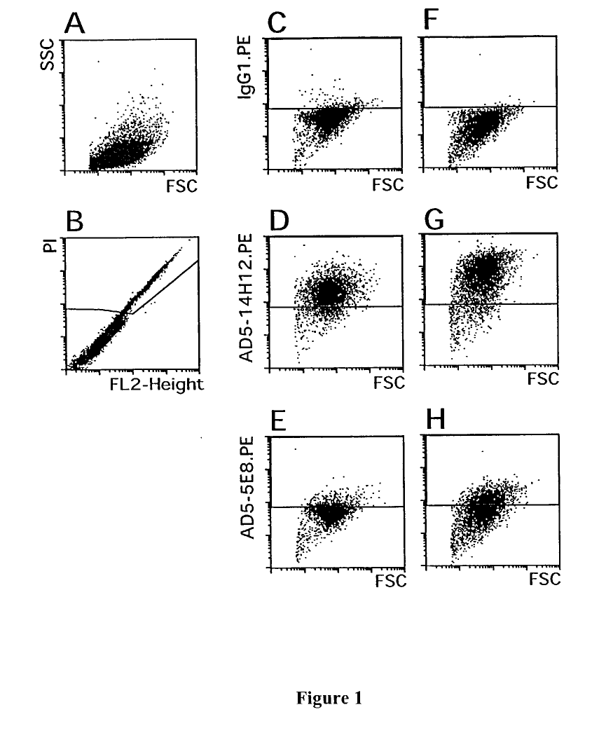

BDCA-3 expressing HD-MY-Z cells (DSMZ: ACC346) were stimulated for 24 hours with 10 ng/ml PMA (Sigma) and 0.5 mg/ml Ionomycin to up-regulate BDCA-3 expression. Cells were cultured at a cell density of 10⁶/ml in medium [RPMI 1640 (Gibco/BRL). 10% FCS (Sigma)] at 37° C. in a humidified 7.5% CO₂-containing atmosphere (FIG. 1). First cells were stained with mouse anti-human BDCA-3 mAbs AD5-5E8 and AD5-14H12 conjugated to Phycoerythrin (PE) (Miltenyi Biotec GmbH). PE-conjugated mouse anti-human CD3 (clone SK7; Becton Dickinson, Mountain View, Calif.) was used for negative control staining. The cells were washed and resuspended in 500 μl PBS/EDTA/BSA (PEB buffer). Stained cells were analyzed by flow cytometry using a FACSCalibur and CELLQuest research software (Becton Dickinson). Dead cells and cell debris were excluded according to their scatter properties and staining with propidium iodide (PI; 1 μg/ml) (FIGS. 1 A and B).

The results are shown in FIG. 1. FIGS. 1C-E shows stainings of unstimulated HD-MY-Z cells, FIGS. 1F-H of PMA/Ionomycin stimulated cells. While BDCA-3 is weakly expressed on unstimulated cells, expression is upregulated upon stimulation.

PMA/Ionomycin stimulated cells were washed twice in 50 mM Tris-HCl pH 8,0 10% Sucrose supplemented with 50 μg/ml Phenylmethylsulfonylfluoride (PMSF), 10 μg/ml Pepstatin A, 2 μg/ml Leupeptin and 2 μg/ml Aprotinin resuspended in the same buffer at 10⁷/ml and ultrasonified at 0° C. (5×6 seconds, 70% output).

Sonified cells were centrifuged at 900×g at 4° C. for 10 minutes to remove nuclei and intact cells. Supernatant was centrifuged at 30000×g 4° C. for 2 hours to obtain purified cell membranes. Membranes were solubilized by incubation in 50 mM Tris-HCl 150 mM NaCl, pH 8,0 supplemented with proteinase inhibitors and 1% IGEPAL CA-630 (Sigma) for 2.5 hours at 0° C. and a cell concentration of 4×10⁷ /ml.

Non-solubilized membrane fragments were removed by centrifugation at 30000×g at 4° C. The supernatant was supplemented with CaCl₂ and MnCl₂ to a final concentration of 1 mM each. The lysate was adsorbed onto a ConA Sepharose column and bound proteins were eluted with 6× column volume elution buffer 20 mM Tris-HCl, 0.5M NaCl, 0.5M Methyl-α-D-glucopyranoside, pH 7.4, 1% IGEPAL CA-630.

(Preparation of AD5-14H12 mAb-column: CNBr-activated Sepharose (Amersham Pharmacia) was washed for 15 minutes in 1 mM HCl (200 ml/g Sepharose) and additionally for 10 minutes in binding buffer (0.1M NaHCO₃, 0.5 M NaCl, pH 8.3). Coupling of 7 mg AD5-14H12 mAb /g CNBr activated Sepharose was done for 14 hours at 4° C. Unbound antibody was washed away with binding buffer. To saturate unbound protein binding places Sepharose was incubated further for 2 hours with 1M Tris at RT. After equilibration in binding buffer the Sepharose was filled into a C10/10-column (Amersham Pharmacia).)

For specific concentration of BDCA-3, eluted proteins were additionally adsorbed onto a AD5-14H12-Antibody-Affinity column and other formerly concentrated glycoproteins without specificity for AD5-14H12 were washed away using 2× the column volume of binding buffer. BDCA-3 was eluted with 15× volume of the column CBP-buffer (50 mM citric acid, 50 mM H₃BO₃, 50 mM Na₂HPO₄, pH 2.5) set onto pH 7 using 1 M Tris solution and then concentrated to a volume of 100 μl using Vivaspin centrifugal concentrator (cut-off 50 kDa (VIVASCIENCE)).

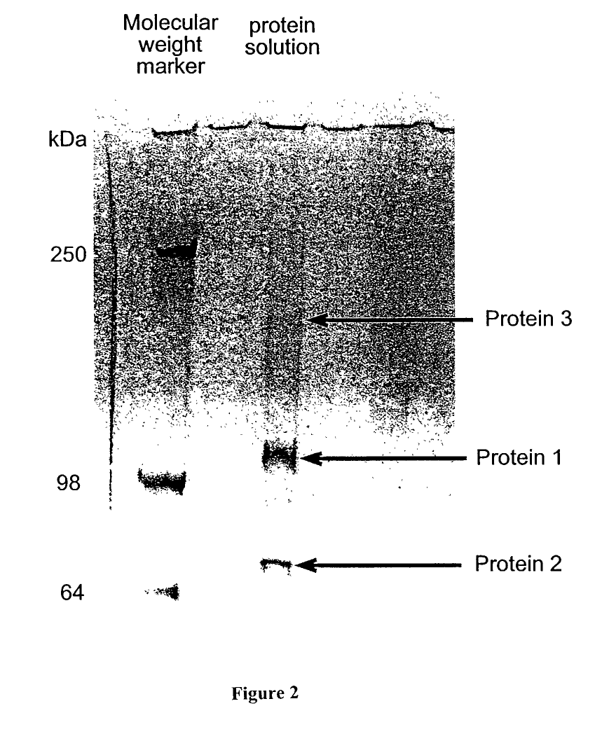

Retained proteins were incubated with 2× Laemmli-Buffer containing DTT and analyzed by SDS-PAGE (6%). To fix the proteins gel was incubated for 45 minutes in 12%(w/v) TCA, and then proteins were stained with Coomassie. FIG. 2 shows the stained Gel.

Three different protein-bands can be seen, with protein 1 (at about 100 kD) corresponding to the previously found band. WO 01/36487. For further characterization the three marked proteins were sequenced with Maldi-TOF at Protagen AG. Obtained results are shown in FIG. 3.

Proteins 1 and 3 were identified as Thrombomodulin (TM; CD141) whereby the band at 200 kDa is a dimer of TM. Protein 2 most likely represents a BSA contamination.

To control the results of Maldi-TOF we generated COS-7 transfectants expressing complete human TM. cDNA for complete human Thrombomodulin (TM) in the expression vector pSR1neo (Conway et al. (1997)) was a gift from Edward M. Conway (Universität Leuven, Belgium). Transfection was done using FuGENE6 Transfection Reagent (Roche) with 1 μg cDNA/4×10⁵ COS-7 cells.

Transfected cells were cultured in RPMI 1640 (Gibco/BRL) supplemented with 2 mM/1 L-glutamine, 10% FCS (Sigma), 100 mg/L Sodium pyruvate (Gibco), 100 U/ml Penicillin (Gibco), 100 μg/ml Streptomycin (Gibco) and 50 μg/ml G418. Expression of hTM was analyzed by flow cytometry after three days of culture. As a negative control, COS-7 cells incubated only with FuGENE6 Transfection Reagent were stained. The following mAbs were used for flow cytometric analysis as described in Example 1: CD141.PE Clone 1A4 (BD Pharmingen); Anti-BDCA-3 Clone AD5-5E8.FITC; Anti-BDCA-3 Clone AD5-14H12.PE; and CD19.PE (Clone SJ25-C1). All these mAbs are mouse IgG1.

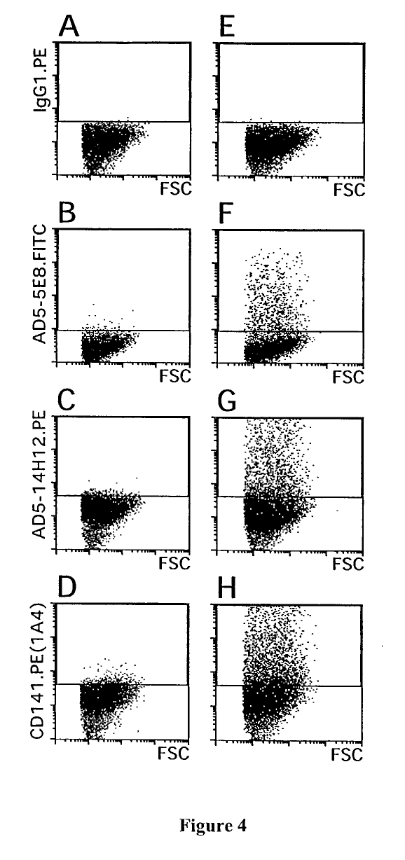

FIGS. 4A-D shows staining of control cells, FIGS. 4E-H of hTM-pSR1neo transfected COS-7 cells. Anti-BDCA-3 antibodies AD5-5E8 and AD5-14H12 as well as the CD141-specific mAb specifically bind to TM-transfected cells (FIGS. 4 F and G) whereas they do not stain the control cells (FIGS. 4 B and C). Isotype control staining with CD19.PE is negative on both cells (FIGS. 4 A and E). This confirmed that BDCA-3 antigen is CD141.

Analysis of Antibody Crossblocking

Co-capping studies as described in WO WO 01/36487 show, that AD5-5-E8 and -14H12 do not bind the same epitope of BDCA-3/CD141.

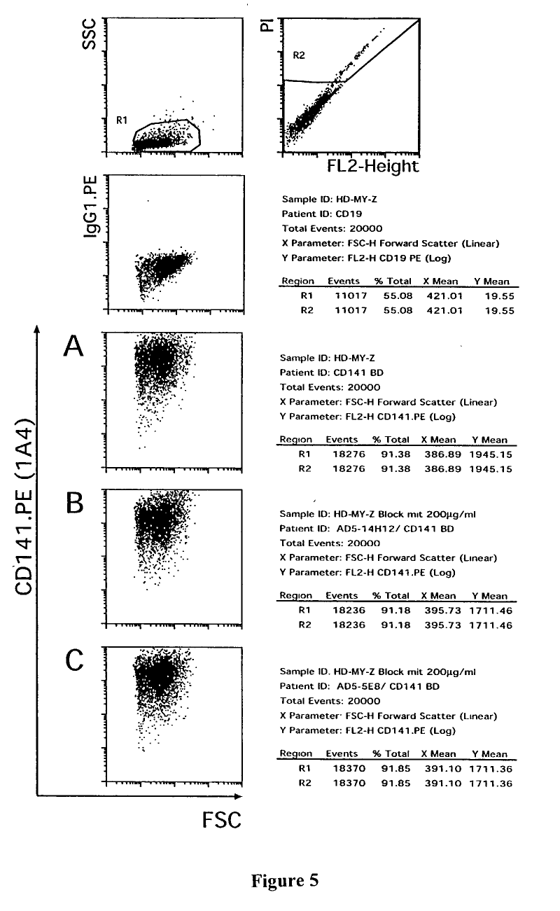

To ascertain whether AD5-5E8 and -14H12 recognize the same epitope as the mAb 1A4 2×10⁶ HD-MY-Z cells were incubated with FcR-Blocking and each of the mAbs AD5-5E8 (FIG. 5 C) or AD5-14H12 (FIG. 5 B) at a concentration of 200 μg/ml for 10 minutes at 4° C. Then cells were stained with CD141.PE clone 1A4 for further 10 minutes at 4° C.

FIG. 5 shows that binding of 1A4 is not blocked by the anti-BDCA-3 antibodies, i.e. 1A4 recognizes a different epitope than anti-BDCA-3 antibodies AD5-5E8 and -14H12.

A. CD141.PE staining of HD-MY-Z with clone 1A4.

B. CD141.PE staining of HD-MY-Z with clone 1A4 after pre-incubation with 200 μg/ml AD5-14H12.

C. CD141.PE staining of HD-MY-Z with clone 1A4 after pre-incubation with 200 μg/ml AD5-5E8.

### EXAMPLE 2

### Isolation of BDCA-3⁺ Dendritic Cells Using Anti Thrombomodulin mAb

BDCA-3⁺ cells were isolated from PBMC by indirect magnetic labeling with PE-conjugated CD141 mAb (1A4) as primary antibody and anti-PE mAb-conjugated microbeads as secondary reagent and enrichment of labeled cells by MACS.

2.5×10⁸ PBMC were resuspended in 1500 μl PEB, 500 μl FcR-Blocking Reagent and 500 μl CD141.PE (1A4) were added and incubated for 10 minutes at 4° C. Cells were washed with 20 volumes buffer, resuspended in 2.5 ml buffer supplemented with 500 μl anti-PE.mAb-conjugated microbeads and incubated for 15 minutes at 4° C. Cells were washed again with 20 volumes buffer, resuspended in 1 ml buffer and proceeded to magnetic separation using LS columns and MidiMACS. To increase the purity, the separation procedure was repeated using a new LS column.

Original and positive cell fractions of the MACS separation were analyzed by flow cytometry as described in example 1. mAbs used for flow cytometric analysis: CD141.PE (1A4); CD14.PerCP (Mø-P9); CD11c.FITC (Ki-M1) and Streptavidin.APC, all from BD Pharmingen. Antibodies used were anti-BDCA-3.Biotin (AD5-5E8) and anti-BDCA-3.Biotin (AD5-14H12).

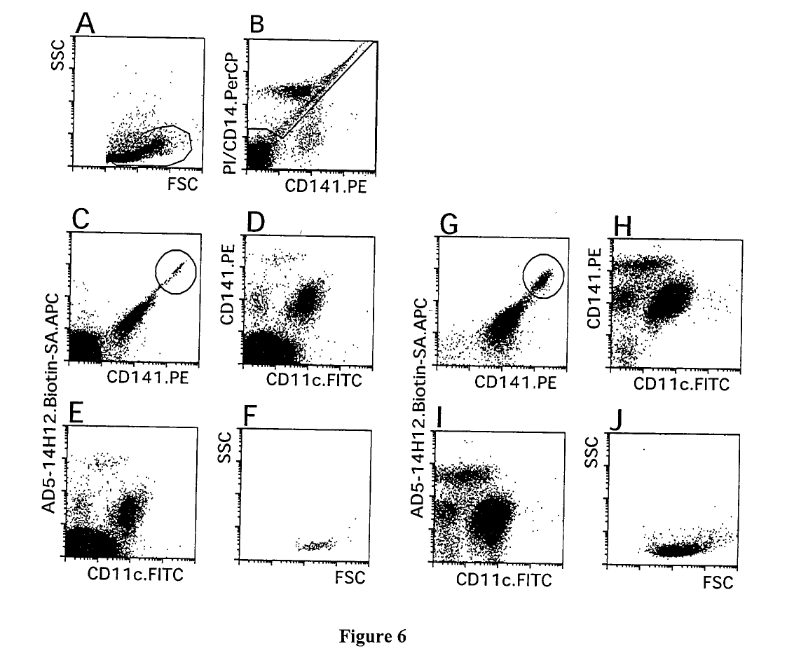

Dead cells and cell debris were excluded according to their scatter properties and staining with propidium iodide (PI; 1 μg/ml) (FIGS. 6 A and B) and CD14⁺ monocytes were excluded according to staining with CD14.PerCP (FIG. 6 B).

FIG. 6 shows staining of cells with CD141.PE versus AD5-14H12 before (C) and after (G) MACS separation. FIG. 6 further shows counterstaining with CD11c.FITC versus either AD5-14H12 before (E) and after (I) MACS separation or CD141.PE before (D) and after (H) MACS separation. Before MACS separation a small population (about 0.09% of viable PBMC) were brightly stained with CD141.PE (clone 1A4 from BD) as well as AD5-14H12 (FIG. 6C, circle). These are CD11cdim (FIGS. 6D & E), CD14- (FIG. 6B), CD1c-, CD123- DCs. Beside this population there are about 17-18% dimly stained cells, which comprise CD14+ monocytes as well as CD¹⁴^{dim} and CD14^{neg} cells (FIG. 6B). The majority of the CD14dim-neg cells is CD11c+, while some cells are CD11c− (FIGS. 6 D & E). After MACS separation CD141bright DCs were enriched to about 4% among viable PBMC and CD141dim cells were enriched to 95%.

### EXAMPLE 3

A magnetic labeling system can be used for positive selection of BDCA-3⁺ cells from human blood. For MACS separation (Miltenyi Biotec), cells are incubated with paramagnetic beads to which an anti-CD141 antibody is conjugated by which BDCA-3 expressing cells are magnetically labeled (see generally Kantor et al., AB; Gibbons, I; Miltenyi, S; Schmitz, J (1998) Magnetic Cell Sorting with Colloidal Superparamagnetic Particles. in: Cell Separation Methods and applications. Marcel Dekker, New York, pp. 153-173; Radbruch, A; Mechtold, B; Thiel, A; Miltenyi, S; Pflüger, E (1994) High-Gradient Magnetic Cell Sorting. Methods in Cell Biology 42: 387-402). Labeled and unlabeled cells are subsequently separated on a column which is placed in the magnetic field of a MACS separator (available from Miltenyi Biotec Inc. 12740 Earhart Avenue Auburn, Calif. 95602, USA). The magnetically labeled BDCA-3⁺ cells are retained on the column, while the unlabeled BDCA-3⁻ cells are collected in the flow through. After removal of the column from the magnetic field, the retained BDCA-3⁺ cells are eluted and can be separated once more over a new column to achieve highest purities.

Isolation of Cells

Peripheral blood leukocytes (PBL) or peripheral blood mononuclear cells (PBM) are isolated by standard methods. For example, to prepare PBL from whole blood by lysis of erythrocytes, start with fresh human blood treated with an anticoagulant (e.g. heparin, citrate, acid citrate dextrose (ACD) or citrate phosphate dextrose (CPD) or leukocyte-rich buffy coat not older than 8 hours). Dilute one volume of cell suspension with 5-10 volumes of lysis buffer (155 mM NH₄CI, 10 mM KHC0₃, 0.1 mM EDTA). Incubate for 5 minutes at room temperature. Centrifuge at 300×g for 10 minutes at 20° C. Wash cells twice by adding buffer and centrifugation at 200×g for 10 minutes at 20° C. Resuspend cell pellet in a final volume of 300 μl of buffer per 10⁸ total cells. For less than 10⁸ total cells, use also 300 μl. For more cells increase the buffer volume to achieve a final cell concentration of 10⁸ total cells/300 μl buffer. Proceed to magnetic labeling. Alternatively, for preparation of PBMC from whole blood by density gradient centrifugation using Ficoll-Paque, start with fresh blood treated with an anticoagulant, e.g. heparin, citrate, acid citrate dextrose (ACD) or citrate phosphate dextrose (CPD) or leukocyte-rich buffy coat. Dilute cells with 2-4 volumes of PBS containing 2 mM EDTA. Carefully layer 35 ml of diluted cell suspension over 15 ml Ficoll-plaque (1.077 density, 20° C.) in a 50 ml conical tube, without mixing both layers. Centrifuge without brake at 400×g for 20-30 minutes at 20° C. Aspirate the upper layer (contains diluted autologous serum that can be used as buffer supplement) leaving the mononuclear cell layer undisturbed at the interphase. Carefully transfer the interphase cells (lymphocytes and monocytes) to a new 50 ml conical tube. Fill up with PBS containing 2 mM EDTA, mix and centrifuge at 200×g for 10 minutes at 20° C. Carefully remove the supernatant completely. Fill up with PBS containing 2 mM EDTA, mix and centrifuge at 200×g for 10 minutes at 20° C. Carefully remove the supernatant completely. Resuspend cell pellet in a final volume of 300 μl of buffer per 10⁸ total cells. For less than 10⁸ total cells, use also 300 μl. For more cells increase the buffer volume to achieve a final cell concentration of 10⁸ total cells/300 μl buffer. Proceed to magnetic labeling. The peripheral blood or buffy coat should not be older than 8 hours.

After the last washing step PBMC may be stored in refrigerator overnight in buffer (PBS, 0.5% BSA, 2 mM EDTA) supplemented with autologous serum.

Additional Steps

Resuspend cells in 300 μl of buffer per 10⁸ total cells (to remove clumps, pass cells through a nylon mesh). Add human IgG to block FcR. Add anti-CD141 conjugated beads. Mix well and incubate for 15 minutes at 6°-12° C. Wash cells by adding 10-20 volumes of buffer (5-10 ml per 10⁸ total cells), centrifuge and remove supernatant completely. Resuspend the cell pellet in a final volume of 500 μl per 10⁸ total cells. Proceed to magnetic separation using a suitable apparatus (see, e.g., Miltenyi Biotec Catalog 2001) with equilibration and washing. The purity of the isolated blood dendritic cells can be evaluated by flow cytometry or fluorescence microscopy (see also section “Instrument and reagent requirements, Optional: Fluorochrome conjugated antibodies”). Stain aliquots of the cell fractions with anti-CD141 monoclonal antibody coupled to FITC or PE. The purity of enriched BDCA-3⁺ dendritic cells may additionally be controlled by counter-staining e.g. for CD14. In this case the BDCA-3⁺ dendritic cells represent a discrete population that is brightly stained for BDCA-3 and negative for CD14, whereas co-isolated monocytes are weakly positive for BDCA-3 and brightly positive for CD14. Co-isolated cells of other blood dendritic cell subsets may be detected by counter-staining for CDc (BDCA-1), BDCA-2 and BDCA-4.

FIG. 7 shows the isolation of BDCA-3⁺ blood dendritic cells from PBMC using the magnetic beads conjugated to monoclonal antibody AD5-5E8 (Dzionek et al., 2000, J. Immunol. 165:6037-46). Cells were separated over two MS Columns (Miltenyi Biotec) and stained with CD14-PerCP and Anti-BDCA-3-PE. Flow cytometric analyses were done by using a FACS-CaliburTM flow cytometer. Cell debris and dead cells were excluded from the analyses based on scatter signals and PI fluorescence.

It is understood that the examples and embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entirety for all purposes to the same extent as if each individual publication, patent or patent application were specifically and individually indicated to be so incorporated by reference.

1. A method of enriching for dendritic cells from a mixture of cells comprising contacting the mixture of cells with an antigen-binding fragment specific for CD141 and selecting the cells that are CD141⁺, thereby producing a dendritic cell-enriched composition.

2. The method of claim 1, wherein the antigen-binding fragment is detectably labeled.

3. The method of claim 2, wherein said selection is flow cytometric selection or magnetic particle separation.

4. The method of claim 1 wherein the dendritic cells are CD1c⁻CD11c⁺CD123⁻.

5. The method of claim 1 wherein the mixture of cells is blood or a blood fraction.

6. The method of claim 1 wherein the blood or blood fraction is from peripheral blood or umbilical cord blood.

7. The method of claim 6 wherein the blood fraction is peripheral blood mononuclear cells, perpheral blood lymphocytes or from a leukopheresis harvest.

8. The method of claim 6 wherein the blood or blood fraction is cultured.

9. The method of claim 1 where the mixture of cells comprises cells from bone marrow.

10. The method of claim 5 wherein the blood or blood fraction is from a subject treated with a agent that increases the number of dendritic cells resident in peripheral blood.

11. The method of claim 10 wherein the agent is Flt3-Ligand or G-CSF.

12. The method of claim 1, further comprising depletion of non-dendritic cell types from the mixture.

13. A dendritic cell-enriched composition obtained by the method of claim 1 or obtained by culturing cells obtained by the method of claim 1.

14. A composition that comprises at least about 5% CD1c⁻CD11c⁺CD123⁻ dendritic cells.

15. The composition of claim 14 that comprises at least about 30% dendritic cells.

16. A composition of claim 14 that comprises at least about 50% CD1c⁻CD11c⁺CD123⁻ dendritic cells.

17. A method of modulating cytokine production by a dendritic cell comprising contacting the composition of claim 13 with an agent that modulates dendritic cell cytokine production.

18. The method of claim 17 wherein the agent is IL-3.

19. A method of distinguishing a subpopulation of dendritic cells from at least one other cell in a population of peripheral blood cells comprising contacting the population with a CD141 binding fragment and detecting the cells to which the binding fragment binds.

20. The method of claim 19 further comprising contacting said population with at least one antibody with specificity for a dendritic cell lineage marker or with specificity for a non-dendritic cell lineage marker and detecting the cells to which the antibody binds.

21. The method of claim 20, wherein the CD141 binding fragment is detectably labeled.

22. The method of claim 21, wherein said label is fluorescent.

23. The method of claim 22, wherein said detection is by flow cytometry.

24. A method of generating an immune response specific for an antigen in a subject, comprising administering to a subject in need thereof dendritic cells from a composition of claim 14, wherein said dendritic cells are presenting the antigen as a MHC-antigen complex.

25. The method according to claim 24, wherein said dendritic cells are genetically modified to express the antigen.

26. The method of claim 24 wherein the immune response is a T cell response.

27. A method of monitoring production of dendritic cell cytokines comprising culturing a composition of claim 14 and monitoring cytokines produced by dendritic cells in the composition.

28. A method of assaying for the effect of an agent on dendritic cell cytokine production comprising contacting a composition of claim 14 with the agent and monitoring the effect of the agent on cytokines produced by dendritic cells in the composition.

29. A method of depleting dendritic cells from a population of peripheral blood mononuclear cells comprising contacting said population with an an antigen-binding fragment and removing cells that are CD141⁺.

30. A method of inhibiting an interaction of a dendritic cell with a ligand that binds CD141 on the dendritic cell surface comprising contacting the dendritic cell with a CD141 binding fragment or reducing the expression of CD141 on the dendritic cell surface.

31. A method of inhibiting an interaction of a dendritic cell and a T cell comprising contacting the dendritic cell with a CD141 binding fragment or reducing the expression of CD141 on the dendritic cell surface.

32. The method of claim 31 that is a method of treating inflammation comprising administering to a subject in need thereof an amount of an agent that inhibits the interaction of CD141 and a T cell or reduces the expression of CD141 on the surface of dendritic cells.

33. The method according to claim 31 wherein the agent is administered in vivo or in vitro.
